(19) United States
(12) Patent Application Publication
(10) Pub. No.: US 2026/0215680 A1
(43) Pub. Date: Jul. 30, 2026

(54) METHOD FOR MULTI-MODAL DIAGNOSTIC

(71) Applicant: Ronen EINAT, Shoham (IL)

(72) Inventor: Ronen EINAT, Shoham (IL)

(21) Appl. No.: 19/144,938

(22) PCT Filed: Jan. 3, 2024
(86) PCT No.: PCT/IB2024/050058

§ 371 Date: Jun. 30, 2025

Related U.S. Application Data
(60) Provisional application No. 63/478,279, filed on Jan. 3, 2023.
(60) Provisional application No. 63/498,249, filed on Apr. 25, 2023.

(51) Int. Cl. A61B 3/10; G01B 9/02004; G01B 9/02091

(52) U.S. Cl. CPC A61B 3/102; G01B 9/02004; G01B 9/02091

(57) ABSTRACT

There are provided methods, devices and non-transitory computer readable medium for Optical Coherence Tomography and Optical Coherence Tomography Angiography solutions. The OCT method is based of interference of electro-magnetic waves on a detector surface. An OCT device includes an interferometer and a broadband wavelength light source featuring short coherence length. The light source beam is split to 2 arms, typically using a beam-splitter, where one arm passes the light onto a reference surface while the other arm projects the light on the sampled object. The back-reflected light from both the reference and sample arms are interfered at the detector surface. Light within the coherence length of both rams would interfere between one another. This interference of light enable reflectivity vs depth mapping to be obtained. OCT interferometry devices usually provide this depth mapping over an area of the inspected sample, that is 3D mapping.

### CROSS REFERENCE

This application claims priority from U.S. provisional patent Ser. No. 63/478,279 filing date Jan. 3 2023, which is incorporated herein in its entirety.

This application claims priority from U.S. provisional patent Ser. No. 63/498,249 filing date Apr. 25 2023, which is incorporated herein in its entirety.

### BACKGROUND

In the field of optical imaging systems there are large variety of modalities, most of these devices are predominantly using one modality or few types of inspection with some variation among the modalities, for example a microscope that changes the illumination angle or wavelength between modalities. One method, named: Optical Coherence Tomography (OCT) is a method for 3D reconstruction of transparent samples and/or topographic characterization of a surface. The OCT method is based on interference of electro-magnetic waves on a detector surface.

For Ophthalmology the eye posterior and anterior diagnostics OCT has become a mandatory diagnostic tool as its capable to detect sub-surface abnormalities that regular imaging methods do not. Current commercial OCT devices in this field are expensive and have large form factor that requires a separate space in the clinic with dedicated desk and in many times need an operator to capture the data.

There is a need to provide a simple, low-cost and small form-factor OCT to enable vast scale usage of OCT commercially and to enable simple and affordable adding of additional imaging modalities using the same imaging optics.

A list of references is provided below:

### REFERENCES

[1] Optical coherence tomography: fundamental principles, instrumental designs and biomedical applications. D. P. Popescu, Lin. P. Choo-Smith, C Flueraru, Y. Mao, S. Chang, J. Disano, S. Sherif, M. G. Sowa. Biophys Rev (2011) 3:155-169DOI 10.1007/s12551-011-0054-7.
[2] En face optical coherence tomography: a technology review. Vol. 10, No. 5|1 May 2019|BIOMEDICAL OPTICS EXPRESS 2177. R. A. Leitgeb. https://doi.org/10.1364/BOE.10.002177.
[3] Common approach for compensation of axial motion artifacts in swept-source OCT and dispersion in Fourier-domain OCT. Dierck Hillmann, Tim Bonin, Christian L\ddot{ }uhrs, Gesa Franke, Martin Hagen-Eggert, Peter Koch, and Gereon H\ddot{ }uttmann. 2012 OSA 12 Mar. 2012/Vol. 20, No. 6/OPTICS EXPRESS 6761. https://www.osapublishing.org/oe/fulltext.cfm?uri=oe-20-6-6761&id=230221
[4] Ultrahigh-Resolution Full-Field Optical Coherence Tomography. Arnaud Dubois, Kate Grieve, Gael Moneron, Romain Lecaque, Laurent Vabre, and Claude Boccara. APPLIED OPTICS/Vol. 43, No. 14/10 May 2004.
[5] Swanson et al (1992)
[6] Fixational eye movements and perception. Michele Rucc, Paul V. McGraw, Richard J. Krauzlis. Vision Research, Volume 118, January 2016, Pages 1-4.
[7] View of What makes a microsaccade?A review of 70 years research prompts a new detection method (unibe.ch). Anna-Katharina Hauperich, Laura K. Young, Hannah E. Smithson. Journal of Eye Movement Research. 12(6):13.
[8] Patent US 2021/0018311 A1. INTERFEROMETRY WITH PULSE BROADENED DIODE LASER. Alexandre R. TUMLINSON; Nathan, SHEMONSKI, San Francisco; Yuan L I U.
[9] Fluorescence Lifetime Imaging Ophthalmoscopy (FLIO) of Macular Pigment. Chantal Dysli, Sebastian Wolf, Mikhail Y. Berezin, Lydia Sauer, Martin Hammer, Martin S. Zinkernagel. Progress in Retinal and Eye Research, Volume 60, September 2017, Pages 120-143.
[10] Fluorescence Lifetime Imaging Ophthalmoscopy (FLIO) of Macular Pigment. Lydia Sauer, Karl M. Andersen, Binxing Li, Rebekah H. Gensure, Martin Hammer, and Paul S. Bernstein. Invest Ophthalmol Vis Sci. 2018 June; 59(7): 3094-3103.
[11] Retinal changes in Alzheimer's disease—integrated prospects of imaging, functional and molecular advances; Veer B.Gupta, NitinChitranshi, Jurre den Haan, Mehdi Mirzaei, Yuyi You, Jeremiah K H. Lim, Devaraj Basavarajappa, Angela Godinez, Silvia Di Angelantonio, Perminder Sachdev, Ghasem H. Salekdeh, Femke Bouwman, Stuart Graham, VivekGupta. Progress in Retinal and Eye Research Volume 82, May 2021, 100899; https://www.sciencedirect.com/science/article/pii/S1350946220300719.
[12] Near infrared spectroscopy in the diagnosis of Alzheimer's disease. Hock C, Villringer K, Miiller-Spahn F, Hofmann M, Schuh-Hofer S, Heekeren H, Wenzel R, Dirnagl U, Villringer A. Ann N Y Acad Sci. 1996 Jan. 17; 777:22-9. doi: 10.1111/j.1749-6632.1996.tb34397.x. PMID: 8624087. https://nyaspubs.onlinelibrary.wiley.com/doi/abs/10.1111/j.1749-6632.1996.tb34397.x,
[13] Twenty-five years of optical coherence tomography: the paradigm shift in sensitivity and speed provided by Fourier domain OCT. Johannes F. DE Bore, Rainer Leitgeb and Maciej Wojtkowski. Biomedical Optics Express 3248 Vol. 8, No. 7, 1 Jul. 2017.
[14] Off-axis reference beam for full-field swept-source OCT and holoscopy. D. Hillmann, H. Spahr, H. Sudkamp, C. Hain, L. Hinkel, G. Franke, and G. Hüttmann, Opt.Express 25(22), 27770-27784 (2017). http://dx.doi.org/10.1364/OE.25.027770.
[15] Kilohertz retinal FF-SS-OCT and flood imaging with hardware-based adaptive optics. Denise Valente, Kari V. Vienola, Robert J. Zawadzki, and Ravi S. Jonnal. Biomedical Optics Express Vol. 11, Issue 10, pp. 5995-6011. https://doi.org/10.1364/BOE.403509.
[16] Wide-field TCSPC: methods and applications. Liisa M Hirvonen and Klaus Suhling. Measurement Science and Technology, 28 (2017) 012003. https://iopscience.iop.org/article/10.1088/1361-6501/28/1/012003
[17]A method and active pixel array for a time-of-flight detection. Patent WO2021255743A1. By Eyal Yatskan and Uri Baror.

### SUMMARY

There are provided methods, devices and non-transitory computer readable medium for Optical Coherence Tomography and Optical Coherence Tomography Angiography solutions.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is an example of a device for OCT;

FIG. 2 is an example of a device for OCT;

FIG. 3 is an example of a method;

FIG. 4 is an example of a timing diagram and an example of a sensing element and four charge accumulators;

FIG. 5 is an example of two dimensional array s of sensing elements;

FIG. 6 is an example of a method;

FIG. 7 is an example of a method;

FIG. 8 is an example of a method;

FIG. 9 is an example of a device for OCT and another modality.

FIG. 10 is an example of a method;

FIG. 11 is an example of decaying signals;

FIGS. 12A-12D are example related to fluorescence signals;

FIG. 12E illustrates an example of a device for determining fluorescence decay information;

FIGS. 13A-13B are examples of a device for OCT and its environment;

FIG. 14 is an example of a method;

FIG. 15 is an example of a device;

FIG. 16 is an example of a device;

FIG. 17 illustrates an example of a device for neurodegeneration detection and tracking;

FIG. 18 illustrates an example of a widespread data collection from edge devices into code;

FIG. 19 illustrates an example of a data management in the database;

FIG. 20 illustrates an example of a wavelength illumination source composes from multiple sources;

FIG. 21 illustrates an example of overlap between slices measured during different measurements;

FIG. 22 illustrates an a method for slice aggregation;

FIG. 23 illustrates an OCT system having a swept source;

FIGS. 24A-24B illustrate an OCT system with a optical distance measurement device;

FIG. 25 illustrates an example of an OCT system that include a distance compensation system;

FIG. 26 illustrates an example of a hand-held portable FF-OCT Device;

FIG. 27 illustrates an example of a segments of a 3D map of an in-vivo tissue with blood vessels;

FIG. 28 illustrates an example of a OCT signals from the segments over time;

FIG. 29 illustrates an example of a phase lines related to the 3D map that are related to the propagation of blood;

FIG. 30 illustrates an example of a method;

FIG. 31 illustrates examples of pulses; and

FIG. 32 illustrates an example of a method.

### DETAILED DESCRIPTION

In the following detailed description, numerous specific details are set forth in order to provide a thorough understanding of the embodiments of the disclosure.

However, it will be understood by those skilled in the art that the present embodiments of the disclosure may be practiced without these specific details. In other instances, well-known methods, procedures, and components have not been described in detail so as not to obscure the present embodiments of the disclosure.

The subject matter regarded as the embodiments of the disclosure is particularly pointed out and distinctly claimed in the concluding portion of the specification. The embodiments of the disclosure, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the disclosure may for the most part, be implemented using electronic components and circuits known to those skilled in the art, details will not be explained in any greater extent than that considered necessary as illustrated above, for the understanding and appreciation of the underlying concepts of the present embodiments of the disclosure and in order not to obfuscate or distract from the teachings of the present embodiments of the disclosure.

Any reference in the specification to a method should be applied mutatis mutandis to a system capable of executing the method and should be applied mutatis mutandis to a computer readable medium that is non-transitory and stores instructions for executing the method.

Any reference in the specification to a system should be applied mutatis mutandis to a method that may be executed by the system and should be applied mutatis mutandis to a computer readable medium that is non-transitory and stores instructions executable by the system.

Any reference in the specification to a computer readable medium that is non-transitory should be applied mutatis mutandis to a method that may be applied when executing instructions stored in the computer readable medium and should be applied mutatis mutandis to a system configured to execute the instructions stored in the computer readable medium.

The term “and/or” means additionally or alternatively.

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent.

Numerous specific details are set forth in the following description to provide a thorough understanding of the invention. However, the invention may be practiced according to the claims without some or all these specific details. To clarity, technical material that is known in the technical fields related to the invention has not been described in detail so that the invention is not unnecessarily obscured.

Reconstruction of transparent samples and/or topographic characterization of a surface.

The OCT method is based of interference of electro-magnetic waves on a detector surface. An OCT device includes an interferometer and a broadband wavelength light source featuring short coherence length. The light source beam is split to 2 arms, typically using a beam-splitter, where one arm passes the light onto a reference surface while the other arm projects the light on the sampled object. The back-reflected light from both the reference and sample arms are interfered at the detector surface. Light within the coherence length of both rams would interfere between one another. This interference of light enable reflectivity vs depth mapping to be obtained. OCT interferometry devices usually provide this depth mapping over an area of the inspected sample, that is 3D mapping. The scanning method over sample area may be a beam scan of a small spot over the inspected surface with single detector using 2D steering optics, such as a rotating mirror that move the spot over the area in a raster line sequence, another scan method use line field with line sensor array, using a 1D rotating mirror or other type of bean steering method for the purpose of covering the sample area. Full field imaging technic does not need for any steering as the inspected area imaged on a 2d senor array where however due some reasons, described below, isn't being used for commercial OCT.

Further to the OCT, most common sample review modes are a white-light source illumination combined with 2D RGB sensor array. Various other imaging modalities exist such as spectroscopy, fluorescence, and polarization. All are obtained by light manipulations using filters, polarizers, and other kinds of light manipulations along with a 2D sensor.

Commercial devices typically not offering both OCT and visible imaging due to the difference between capture modes (scan vs 2d imaging). There are some devices that are having this offering however they employ different internal optics for the different modalities. As a result, these devices are large and expensive.

There are provided some Full Field OCT (FF-OCT) methods with simplified components, diffraction limited resolution, shot noise limited SNR and unbounded depth range. The imaging optics can have multiple use for OCT and other imaging modalities such as spectroscopy, fluorescence, polarization and others. Giving rise for low-cost small form factor device. There are some prior arts for methods comprising Full-Field OCT that utilize field illumination and imaging optics, some are not fit for in-vivo testing, some having complexity and/or price disadvantages. A Time-Domain Full Field OCT (FF-TD-OCT) example was provided with shot-noise limited signal quality, using a phase modulator. A major disadvantage of this work was the use of 4 scanning sweeps for the complete demodulation of the signal to get the interference modulation information. The reference light path was modulated by a phase modulator at high frequency that produces the interference modulation. The light source was flashed with pulses of light with the same frequency for ¼ cycle time different phases. The phase modulation combined with synchronized light flashes results that the sensor collecting light only at flash time during its exposure time. Complete modulation characteristic measurements 4 different phases used between the light source and phase modulation, utilizing 4 different scans of the sample. For a stationery sample this method is adequate. However, for live a moving sample it would pose noise to the modulation amplitude measurement as each scan differs from one another due to motion of the sample vs the sampling system.

A Swept-Source Full field OCT (FF-SS-OCT) was provided, utilizing Adaptive Optics (AO) for aberration correction, claiming for high resolution image quality over diluted eye, in vivo scanning. The configuration utilizes a Swept radiation source and field imaging and the use of fast capture sensor at very high rate. The high spatial resolution obtained with high NA optics exhibit low DOF thus offering depth data only over optics DOF. This will be further discussed below. Also, the swept radiation source is an expensive component. Contributing to overall higher size and price.

Specifically for ophthalmology the OCT analysis has become crucial tool for eye diagnostics, its ability to generate a 3d mapping of the retina enable diagnosis of eye diseases that are not shown with regular imaging methods. The OCT provides information about retina layers, with good layer separation and abnormalities detection for most popular diseases such as Age-related Macular Degeneration (AMD) Glaucoma and others. OCT also is use for Diabetic retinopathy detection and tracking, Diabetic patients are regularly being monitored with OCT for detection of signs of retinal deterioration.

Eye review, especially the retina, is becoming relevant for neurology as the eye is part of the brain neurology. Research frontline has shown indication that the retina is affected from neural degradation of dementia patients in parallel to the brain and retinal inspection can provide bio-indication for the early start of dementia diseases such as Alzheimer Disease (AD) This research branch is growing and expected to yield the need for wide distribution of retinal imaging devices with multi-modal capabilities, including OCT. Many eye diseases such as Age-related Macular Degeneration (AMD), Glaucoma and diabetic retinopathy are chronic so there is a need for regular monitoring of patients for years, same as the need to monitor people for neurodegeneration and diabetic retinopathy signs as early as possible for treatments and tracking of the disease progress over time that will provide feedback of treatments. Given the growth of population and increasing numbers of dementia disease it's clear the need for large distribution of sample device availability to the populations. In addition, the data captured of the devices may become huge and need to be handled adequately to enable review, remote diagnosis, auto-diagnosis and new diagnosis development.

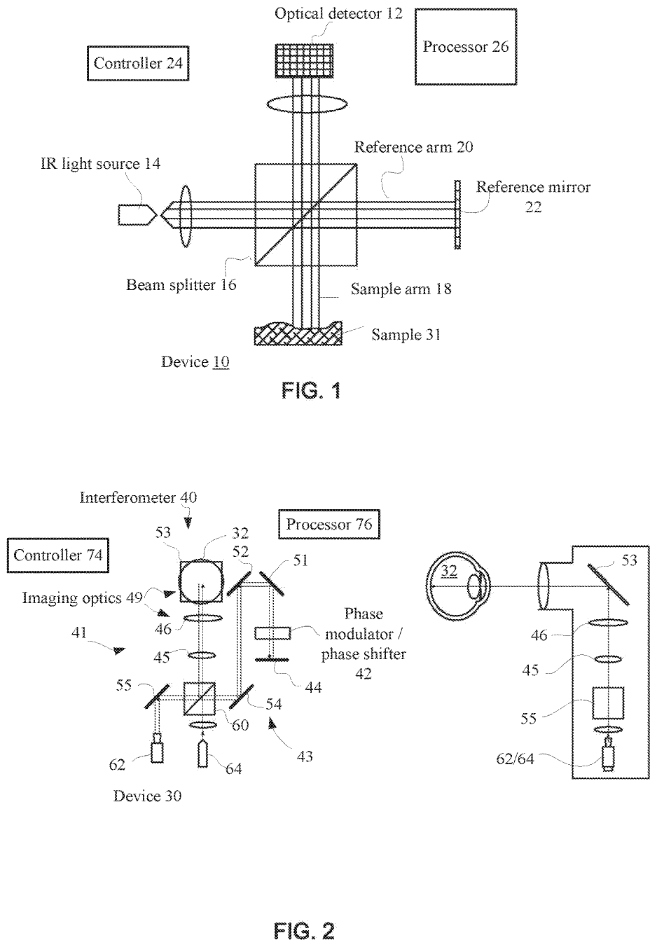

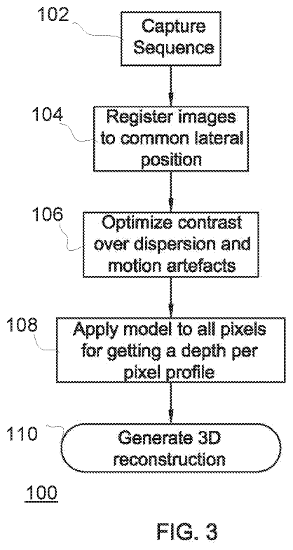

FIG. 1 illustrates an example of an OCT device 10 that evaluated sample 31. A broadband light source beam (from IR light source 14) is split by a beam splitter 16 into sample arm 18 and reference arm 20 (terminated at reference mirror 22), reflected light from both arms interfere on the detector 12 surface via the beam splitter. The OCT device has a controller 24 and a processor 26.

There are various methods for OCT implementation. Time Domain OCT (TD-OCT) uses a wideband light source with short coherence length. In this mode the reference optical path length is typically modified by motion of a reference mirror. This motion changes the depth of the coherence window such that for each length results an interference with different depth of the sample. This way the movement of the reference mirror can be used to scan the desired depth range. The first OCT systems used this method to 3d reconstruction of a sample reflectance over depth. The scan over depth called “A-Scan”. These first devices used a fast-moving mirror to complete A-Scans over the sample at certain point illuminated by small spot of the light. After the first A-scan, the spot moved laterally (or the sample is being moved) to next nearby location where next A-Scan is being performed. This way a 2d area can be scanned for 3d reflectance mapping.

The fast motion of the reference arm during A-scans modulates the interference pattern over the detector surface such that the detector circuit measures it's modulation amplitude in a frequency related to the motion velocity of the reference mirror. The temporal modulation amplitude in each time/position gives the sample reflectance in each depth. If a sample is with high reflectivity the returned optical electric field on the detector surface is high, thus the interference modulation amplitude would be high. Similarly low reflectance from the sample in a position corresponding to the reference arm path length will result low modulation amplitude during scan motion. Spectral Domain OCT (SD-OCT) evolves from the Time-Domain OCT where the light source broadband is being used to replace the TD moving arm. The broadband light is being split to wavelength sections, using a grating or a prism prior to the detector, where each detector is sensitive to a different range of wavelengths. The set of signals obtained from the different wavelength sections passes a furrier transform to result the desired reflectance over the whole depth range. This method replaces the need for fast moving reference mirror. The SD-OCT method is commonly quoted as improving signal quality by 1 to 2 orders of magnitude while increasing throughput. Another method for Spectral Domain OCT, is Swept-Source OCT (SS-OCT) in this case the light source changing its center wavelength as function of time. Thus, the split to spectral slices is done over time instead of spectral split in space. SS-OCT also requires Fourier transform to get the depth data.

OCT tools typically generate a 3d mapping of a region of the observed sample, that is the 3d mapping is done over lateral region, the depth axis is called “Axial” and the lateral directions are transverse axes. The axial resolution determined by the light source spectral band given by:

$${D}_{z} = \frac{2 ln 2 {λ}_{0}^{2}}{π Δλ} \qquad ( 1 )$$

The transverse resolution is determined by the optical resolution of the detection optics. There are few options for performing the xyz scanning, where z- is axial, x and y are lateral (transversal) scanning. In many cases the scanning of the surface is obtained by single spot scan into single detector where a 2d mirror steer the beam of light to scan the spot laterally over the sample. In each position an axial scan is done, one after the other.

There is provided a low-cost edge device, enabling widespread distribution for capturing retina or a sample imaging with various modalities using a single 2d sensor. The devices can be a part a system for storage, share, developments and many more services described below.

Another embodiment extends the imaging capturing device to include other biosensors such as EEG of the brain to contribute additional complementary data to further enhance detection specificity and sensitivity.

There is provided a low-cost edge device, enabling widespread distribution for capturing retina or a sample imaging with various modalities using a single 2d sensor. The devices can be a part a system for storage, share, developments and many more services described below.

There is provided a device performing multi-modal sensing capabilities, comprising high quality OCT and various other imaging modalities using the same imaging optics. The device can be widespread distribution and cloud-based data collection from the distributed devices for multi-sensor fusion, data mining, sharing, diagnosis and AI-based diagnostics development.

There is provided a OCT mode that enable Full-Field (FF) imaging. Meaning the capture of 3D data obtained by a series of 2D images of full-field imaging using 2D sensor array. The term “full-field” relates to 2D field imaging onto 2D sensor array without scanning with beam steering. Regarding OCT implementation there are 2 main methods. Time Domain OCT (TD-OCT), and Spectral Domain OCT (SD-OCT) also commonly named Fourier Domain OCT (FD-OCT). There are some advantages for each method. The SD-OCT has a better SNR, and historically improved scanning throughput since it eliminates the need for fast axial scanning mirror in the reference arm. However, in the case of FF-OCT using time-domain imaging the motion of the reference mirror can be slow as it scans all image at once over depth, thus the fast axial scanning not needed. If one desires FF imaging for cost and simplicity reasons than SD-OCT would need to be designed with Swept radiation source (SS) illumination, since it's difficult to implement with broadband illumination and grating/prism+FF. The SS is a light source that have single wavelength at a time and the wavelength changes along time, where the 2D sensor captures the signal wavelength in time, hence called swept source. Each capture of different wavelength. The SD-OCT and SS-OCT also have some disadvantages such as dispersion, fall off, auto-correlation noise and the need to perform a Fourier transform over the data to obtain depth info. In the below description it will be discussed in more detail.

Further modalities can be spectroscopy or fluorescence using illumination at one wavelength and receiving images at another wavelength, depending on desired sampled feature. The fluorescence capture implemented using filters both at the illumination and the collection, another option is to use narrow wavelength illumination light source and a blocking filter in the receive channel to block out the illumination and receive the other wavelengths due to fluorescence.

Additional modalities that can be implemented are Fluorescence Lifetime Imaging Ophthalmoscopy (FILO) or Fluorescence Lifetime Imaging Microscopy (FILM). As the emitted fluorescence light decay time of biological matter contains important information of the concentrations and type of molecules contained. Moreover, the decay time may exhibit 2 or more profiles that are very important for material distinguishing as each material have different decay time. In this invention we utilize the multi-node sensor fast switching time+a sequence of phased capture to produce decay profile that will enable FLIM or FLIO of at least 2 time constants using the same imaging optics. Along with fast pulses activation illumination sync' with the sensor detection phases.

An imaging device with multi-modalities using the same imaging optic path provide the following advantages:
a. Simple and cost-effective design.
b. Easy usability, fast capture. Once in alignment with the sample, all modalities can be activated for capturing. No need for sample capture using multiple devices
c. Smaller form factor, small device.
d. Alignment between modalities that simplify sensor fusion diagnostics, since the same imaging optics used for all capture modalities, features captured by all modalities are aligned accurately one to the other, providing powerful diagnostic information for further analysis of the inspected surface. The only difference might be due to chromatic aberrations.

The device further has a processing unit for data analysis, feature fusion algorithms between the various modalities, diagnostics detection. A synchronization and control logic to activate the modes without interfere one another. Also having a connection with a computer for transfer of data and results to the main computer.

Device wide distribution in the populations will also enable wide data collection in a cloud or dedicated database storage. For sharing, tracking over time, and research analysis and development of new diagnostics.

### Swept Radiation Source FF-OCT:

There may be provided a Swept radiation source FF-OCT (FF-SS-OCT), as described above, the wavelength of light is swept during exposure and capture sequence, such that at each time the light source would emit a single wavelength, thus each detector exposure would accumulate charge from one specific wavelength. Subsequent exposures capture different wavelengths, this way spectral split into ranges obtained via the time variant wavelengths change where SD-OCT use spectral split of a broadband light source using a grating or a prism. Since the illumination wavelength changes in time, it is possible to implement full field imaging. SS-OCT is a kind of FT-OCT, where the data captured over spectral slices need to pass Fourier transform. SS instantaneous band is narrow, thus obtaining wide coherence length and enable wide depth ranging.

FIG. 2 illustrates the basic FF-OCT device of an embodiment. Both SS and TD configurations presented. The device optical path is folded to provide thin device width. The figure depicts a plausible implementation for Ophthalmology where the eye retina is being reviewed. For the SS case, the image capture sequence contains a series of exposures of the 2D sensor that may be a global shutter CMOS sensor, each exposure is in synchronization with illumination flash of the swept source. Exposure is typically followed by a read-out time from the sensor to the processing platform, during read-out light can be switched off. At the proceeding exposures the illumination wavelength swept over range of entire spectral bandwidth. Some sensors feature parallel exposure and readout, where during the exposure time of the one image the previous exposure data is sent to the platform, this enables to increase the active duty cycle thus reduce scan time. A series of images are being collected. It should be noticed that the wavelength value can be sequential per capture from low to high or vice versa, but it can also be arbitrary arranged with non-constant step size between flashes.

FIG. 2 illustrates an FF-OCT device 30 (denoted device 30) and a sample such as eye 32. The device includes radiation source 62, sensing unit 64, beam splitter 60, interferometer 40 that has sensing arm 41 and reference arm 43, phase modulator or phase shifter 42 (shown in the reference arm but may be located in the sensing arm), tuneable focusing optics 45, imaging optics 49 that includes lens 46 and folding mirror 53, reference mirror 44, and additional reflecting mirrors 51, 52, 54 and 55. The radiation source 62 may be a swept radiation source or a broadband source. The sensing unit 62 may include one or more 2D array s of sensing related elements. Controller 72 controlles the device. Processor 76 may receive detection signals and process them. FIG. 2 includes a detailed front view (left side of FIG. 2) and a side view that illustrates only some of the optical components of device 30.

### Dispersion and Motion Artifacts.

Spectral Domain OCT is very sensitive to axial errors as they are magnified by a factor of A/AA that can be as high as 10-20. Thus, in cases where the optical path of the sample arm changes relative to the sample arm along wavelength change scan swipe it will result quality reduction of the depth reconstruction. Path length difference can be caused due to dispersion effect as the light passes through sample material, such as the eye fluids, and/or distance variations in time due to mechanical vibrations. Below we describe the causes and their resolutions.

Dispersion: The dispersion relates to the change of the refractive index of the sample as a function of wavelength. A biological sample such as the eye causes the optical path length to change due to refractive index change in the cornea, lens, and eye fluid. As a result, the obtained depth mapping after Fourier transformation is distorted since the reference optical path does not have the same optical path over wavelength as the sample arm.

Motion Jitters: Another cause for signal quality degradation is motion vibrations between the device and the sample as the sample might not be tightly fixed to the device there may be micro-vibrations that change the optical path length during the scan. This change, like dispersion causes quality degradation. During the time of scan, e.g. 1 second capture sequence, these mechanical vibrations change the optical path.

These dispersion and motion-based distortion can be compensated; however, we need the exact index variation over wavelength mapping and motion details, but there may be variations sample to sample, and the vibrations are not known at the time of capture. It was shown that the effect of difference of the optical path between the ref arm and the sample arm, dispersion, vibrations or other cause can be characterized as a phase delay to the interference correlation function that is e^{-ikf(*)} where ƒ(k,t) is the residual phase delay from zero delay at the scan start and k is the wavenumber and t is time from start of scan. Thus, compensating the effect can be done by multiplying the data samples by its complex conjugate e^{ikf(*)}. There are several approaches in the literature for performing this compensation. Below described compensation optimization method for contrast enhancement. For simplicity we assume no lateral jitters during scan, that is the pixel (x₀, y₀) represent the signal obtained of a small area on the sample where this area is constant during scan.

The dispersion and motion-based distortion effect may be separated from the data itself by means of parametrical modelling of the variations using contrast enhancement methods by the following:

$$R ( {x}_{0} , {y}_{0} , z ) ∝ ℱ \{ S ( {x}_{0} , {y}_{0} , {l}_{i} ) \} ; \qquad ( 2 )$$

$$C ( {x}_{0} , {y}_{0} ) = {Contrast}_{over z} ( R ( {x}_{0} , {y}_{0} , ) ) ;$$

$$dispf = {max}_{over a , b , c} [ {Contrast}_{over z} ( \hat{R} ( {x}_{0} , {y}_{0} , z ) ) ] \qquad ( 3 )$$

$$and : \hat{R} ( {x}_{0} , {y}_{0} , z ) = {ℱ}^{- 1} \{ S ( {x}_{0} , {y}_{0} , {l}_{i} ) \cdot {e}^{i k f ( {l}_{i} , a , b , c , … )} \} \qquad ( 4 )$$

S( ) is the normalized signal obtained in the detector at (x₀, y₀) for the {lᵢ}sequence of wavelength frequencies of the light source. And R( ) it's the calculated reflectance square root of a pixel located at (x,y,z) for the z depth. [symbol] ⁻¹( ) denotes the Inverse Fourier Transform (IFT) function. The term \hat{R}(x₀, y₀, z) is the reflectivity mapping obtained for the enhanced contrast. The term e^{ikf(l}^{ᵢ}^{,a,b,c . . . )} is the conjugate factor for dispersion and motion compensation. The function ƒ(lᵢ, a, b, c . . . ) is a model based function over lᵢ with parameters a,b,c . . . , ƒ(*) can be a polynomial function or any other model based function that is being optimized for obtaining the best contrast over z axis. As Its expected that the best signal quality conditions would be obtained once dispersion and motion compensation is optimal. The maximization process of signal contrast is a search among the model parameters for a,b,c . . . value that will result the desired best contrast. The model type and parameters should be selected such that it will represent the dispersion and motion variation that is expected to be somewhat smooth over wavelength and continuous. Also, the model parameter numbers should be low. There are many optimization methods, as exhaustive search or heuristic such as steepest decent.

The above-described method for signal quality improvements defined for one image pixel termed (x₀, y₀) ideally one should repeat this operation for all pixels in the detector array. This might be computational intense, long duration and costly. However, both dispersion and motion can be assumed common for the sample or at least for some regions so the compensation calculator for a pixel or a group of pixels may be distributed to their neighbouring pixels. This way the compensation optimization process can be done on small sample of the data but would be used to the depth reconstruction of all pixels. Averaging over group of sample pixels in each zone also helps to reduce temporal noises. The depth generation over pixel array flow is:
Split the image to G×H zones.
Per zone, select sample pixels for the optimization process.
   Perform the compensation process described at eq (2), (3) & (4) above for the zone sample pixels.
   Find best contrast model ƒ_{g,h}(lᵢ, a, b, c . . . ); g=1 . . . G, h=1 . . . H. By averaging over calculated compensations for each of the sample pixels. Another option is the average the signal of sample pixels followed by compensation calc.
Distribute the compensation model to all pixels in the image. Each model can be used to calculate depth mapping of its own zone, a better approach may be to interpolate the compensation phase between zones. Interpolation will reduce error for pixels near the borders between zones. Also, it will guarantee spatial continuation.

### Fall Off Issue and Solution.

The Spectral Domain OCT splits the illumination wavelength range into slices of small spectral ranges by using a grating or prism. Each detector receives a sub-range. The effect of this range causes for degradation of contrast as the function of depth, since it's not a single wavelength. So features in proximity to reference OPL distance will have higher contrast, where the further the delta distance increases from the reference distance contrast being degraded.

This issue named “full-off” appears also at SS-OCT based systems, as the source wavelength change continuously during the temporal dada acquisition, typical spot or line field scanning methods need to sweep the wavelength range fast and continuous as the detector analog signal is being sampled in time. Such that each sample also contains a slice of spectral range.

There is provides a Full Field SS imaging in which the source wavelength changes slowly over time using discrete exposures, so it's viable to implement stepwise wavelength changes over time, in synchronization with the 2D detector array such that each exposure image capture will contain a single wavelength, or very narrow range, rather than range slice as would be the case for linear continuous wavelength sweep example.

### Lateral Jitter Fix—(Eye Fixation Jitter).

During scan capture, lateral jitters may degrade imaging quality as each image capture in the 2D sensor might suffer from some miss-alignment due to lateral (transverse) jitters. As a result, the reconstructed 3D mapping might degrade contrast and sharpness. Depending on the jitter's amplitude and speed of motion. At Ophthalmology, the eye, once fixated on an object, exhibit some jitters, where its optical axis moves over time. This motion called “Eye Fixation Jitter” is an issue if one wants to capture an image of the retina or the exterior part of the eye. There are 3 basic types of eye motions:
1. Microsaccades, typically 2-3 times/sec 20-80 arc min.
2. Optical drifts—low frequency random walk <40 Hz
3. Tourmore—High frequency random walk 40 Hz<f<100 Hz. Typically lower in amplitude vs Microsaccades and Optical drifts.

Among these 3 types, the microsaccades have highest amplitude and may introduce high smearing noise that causes contrast loss over sensor's 2D pixel array image. It was shown that a duration of ~1 ms is safe for stable capture of the eye for single exposure without these smearing effects. This is also applicable once motion jitters are considered. For FF-OCT case, where the scan process includes multiple exposures, these jitters might drastically affect image integrity, as the different images would not have fully aligned one another. Yet having capture of multi-frames where each exposure is shorter than 1 ms the images would not suffer from blurring artifacts. However, jitters induced lateral displacements can be compensated by registration process among captured image frames. However, as explained, each image of certain depth does not contain motion artifacts even if the whole capture sequence may be 1-2 sec.

### DC and Side Ambiguity Resolving

For a case transparent sample where the internal depth data is of interest the basic SS-OCT method can't resolve the ambiguity of depths on the 2 sides of the 0 (zero) OPL difference as the FT of a real signal exhibit symmetry around the center zero frequency. One way SS-OCT systems resolve this issue by using only ½ of the coherence sides. That is to set the zero OPL difference outside of the sample. This solution is undesirable for our implementation as narrows the work range and limits the possible depth range. Another way to resolve this issue by a phase modulation of the reference arm of π/2 (λ/4) cycle used.

Fourier Domain OCT is based on spectral interferometry, where recombined light from reference and sample arms is spectrally separated. The detected interference signal as function of k (k=2π/λ) may be expressed by:

$${I}_{cos} ( k ) = {I}_{r} ( k ) + 2 \sqrt{{I}_{s} ( k ) {I}_{r} ( k )} {\sum }_{n} {α}_{n} cos ( k {z}_{n} ) + {I}_{s} ( k )$$

Where Iᵣ(k) and Iₛ(k) represent the intensities reflected from the reference arm and the sample arm respectively. a, is the square root of the sample reflectivity at depth zₙ where at z=0 is the sample z that is equal to reference arm OPL, the power spectra of IFT over I(k) would result ambiguous (zₙ, −zₙ) solution since the intensity is real number as the nature of FT. Adding a swipe over k with the addition of π/2 phase delay in the reference arm or sample arm would result:

$${I}_{sin} ( k ) = {I}_{r} ( k ) + 2 \sqrt{{I}_{s} ( k ) {I}_{r} ( k )} {\sum }_{n} {α}_{n} sin ( k {z}_{n} ) + {I}_{s} ( k ) \qquad ( 13 )$$

The combination of I(k)=I_{cos}(k)+iIₛᵢₙ(k), where i represent the imaginary part will produce a complex representation that will result a unique zₙ at the inverse FT transformation. However, this requires a second set of frames to be collected with the phase shifting.

Another issue is the DC component (at z=0) of the FT, as the Iᵣ(k) and Iₛ(k) parts can't be removed, traditional SS-OCT devices utilize balanced detection that removes the DC component of the signal. Allowing only the coherence signal for the capture, another approach is to subtract the reference spectral density profile form the interference density profile.

A different approach for DC component and complex conjugate (negative z) removal proposed at the literature by use off-axis angle between the sample and reference arm optical axis towards the sensor. This angle produces spatial fringes on the sensor surface that corresponds to the off-axis angle, the wavelength and the coherence amplitude. These spatial fringes used for the DC and complex conjugate suppression. It was also shown that this method reduces autocorrelation noise (will be described below). The spatial fringe rate on the sensor surface is used by spatial or frequency filtering to extract the desired information. However, the method limits spatial work range considerably (by ~⅓) thus resolution affected and requires detection spatial response to be much higher than for on-axis method that is complex and costly.

### FF-SS-OCT Full Processing Flow

Summarizing the above discussion, the processing flow 100 presented at Error! Reference source not found. starting with a capture sequence 102 of multi-images of the sample, each image capture of short duration that would ensure no blurring due to motions of any kind. Following the capture sequence, a registration step 104 is done for laterally align the images, that would compensate the vibrations and jitters in the lateral directions (x,y), after the registration the next step is to compensate for dispersion and axial direction vibrations 106 as described above. This may be followed by step 108 of applying a model to all (or some) pixels for getting depth per pixel profile, Following that—a 3D reconstruction can be made—see step 110.

### FF-SS-OCT Enhanced Resolution.

The OCT axial resolution determine largely by the spectral width of the light source as stated at eq. (1) done simply by increasing the swift range of the source, however some issues may arise such as the overall response of the sensor over the bandwidth range may not be equal over range such that it would influence the obtained reconstructed mapping, also dispersion effect more pronounce, however these issues may be resolved mathematically as known in the art and described above for dispersion compensation.

Regarding the lateral resolution. There is an inherent tradeoff between Depth of Focus (DOF) and resolution that contradict each other. The higher the spatial resolution the lower the DOF of the optical imaging system. Thus, improving the lateral resolution ultimately reduces DOF since they both relate to the optical Numerical Aperture (NA).

### Diffraction Limit Resolution Determined by:

$$Δ x = \frac{4 λ f}{π d} = 1.27 λ {f}_{\#} \qquad ( 5 )$$

Where λ is the wavelength [m], ƒ is the focal length and d is the optical aperture diameter [m]. F_{#}=ƒ/d is the F-Number that is inversely proportional to the NA.

The DOF determined by:

$$DOF \approx \frac{2 {u}^{2} c}{{f}^{2}} {f}_{\#} = \frac{2 {u}^{2} c}{fd} \qquad ( 6 )$$

Where u is the distance to subject [m], c is circle of confusion [m]. Increasing the aperture d increases the lateral transverse resolution but reduces the DOF. In this invention embodiment we can repeat capture sweeps 2 or more times. Each sweep is done with different reference path length and different focusing distance of the sample such that the entire depth is scanned.

### Example

Suppose we want to map the depth of a sample in a range of 2 mm, meaning we must set DOF=2 mm. to maintain focus over range.
Also assume:

λ1 - 900 nm - Central Wavelength.
ƒ - 20 mm - Focal length.
u - 30 mm - Target distance
c - 15 um - Circle of confusion.

The obtained required aperture (eq 11) is d=0.675 mm
Using (5) the axial resolution would result: dx=33.9 um.
Improving lateral resolution to 10 us, Forces the DOF to be =~0.6 mm. Thus, in single sweep, DOF to resolution tradeoff forces either axial range decrease or low resolution. For this invention we can win both by multi-sweep, each sweep scans different depth ranges by the following:
   1. Set the aperture d to the desired dx resolution.
   2. Set the opt' path length of the reference arm to match the length of the start of the desired depth range.
   3. Set the imaging focusing distance to the same depth as the reference path length is set to.
   4. Sweep source and capture images to scan the spectral range.
   5. Move reference path length and focusing to the next depth slice, typically by a measure of 1 DOF.
   6. Repeat Sweeping again for the next slice.
   7. Continue slice sweeping steps 5 and 6 until the entire depth range is covered.
   8. Final 3d representation can be obtained from combining the slices maps to each other. May need smart registration step to align the slices to each other.

Thus, by splitting the depth range to sub ranges (slices) we can improve lateral resolution while having larger depth work range than can be obtained in single sweep.

Noting also that increasing the aperture d of the optics not only improve resolution but also increases the collected light power from the sample and reduces speckle noise in the captured sample.

### Full Field TD-OCT:

Another embodiment is Time-Domain Full Field OCT (FF-TD-OCT), also using a 2D sensor array for direct imaging of the retina over the sensor surface without the need 1D or 2D scanning method. The light source used in this method is wide band, typically in the NIR range. Referring to FIG. 2. a phase modulator in the reference arm modulates the phase of light between 2 phases about λ/4 amplitude of the center wavelength, obtaining modulation p-p of ~λ/2, alternating between 2 discrete phases, sinusoidal or any other shape that alternates these phases. The phase modulation of the reference arm light returning to the sensor is than positively and negatively interfere with the back propagated sample arm light, but only the portion of sample light that returns from the same optical distance as the reference arm within the coherence window. Modulation amplitude at the sensor indicates the reflectivity of the sample at the coherence window distance. The reference optical path is modified as the motion of mirror changes the optical path, causing the coherence distance in the coherence depth in the sample to change accordingly.

FF-TD-OCT with Multi Node Sensor:

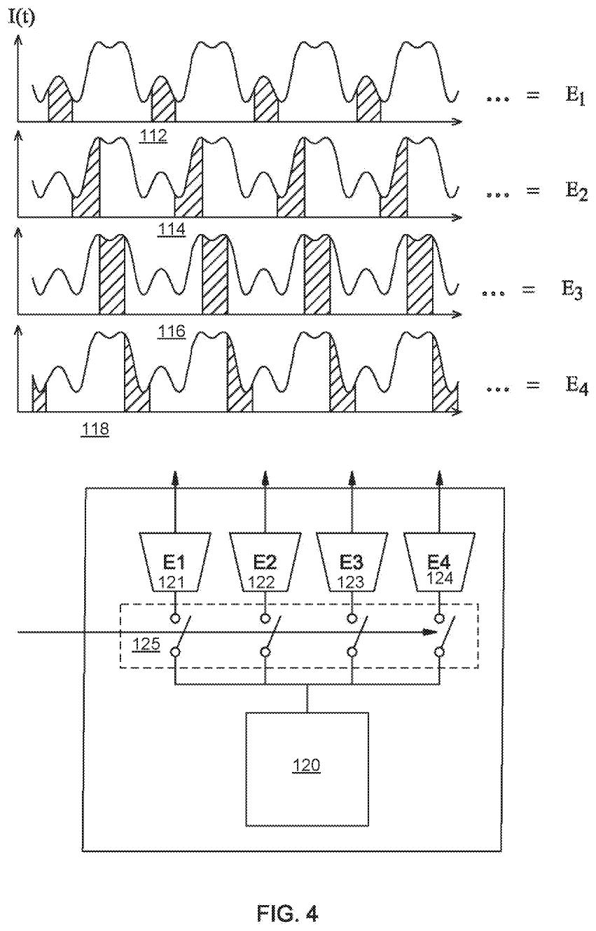

In this invention, one embodiment, we implement FF-TD-OCT by using a multi-node 2D sensor, a multi-node sensor is a sensor that can capture and accumulate optical energy into plurality of accumulation buckets (nodes) during exposure time. The collected photoelectrons at the photodiode (PD) in the pixel active area can be accumulate into a plurality of nodes in accordance to control signals receive in the pixel that direct the collected charges to one of the nodes. FIG. 4 shows 4 node pixel exposure to collect 4 cycle parts of π/2 each.

FIG. 4 illustrates a Multi Node pixel structure explanation. Image a illustrates a cyclic signal capture, each bucket (node) (of four buckets 121, 122, 123 and 124) collects light energy at different exposure time (see different dashed areas in timing diagrams 112, 114, 116 and 118)—using switching unit 125 to distribute the charges between the bucket, in this case the buckets are set to collect integrated charge in the PD 120 of different time interval such that each one gets ¼ of the modulation cycle. A pixel structure principal of operation shown where the charge collected in the PD passed to one of the buckets E1 . . . E4 in accordance to the control lines in the pixel.

The sensor activated in homodyne receiving mode such that each node exposed to a phase of the modulating frequency for multiple modulation cycles and aggregates the photo-electrons at the node for many cycles, number of accumulated cycle can be arbitrary selected as there is no theoretical limit.

For Example—using 4 nodes sensor, overall sensor exposure time may be 1 ms and the phase modulation frequency is 100 kHz. So, within exposure time there would be 100 modulation cycles. The buckets (nodes) E1 . . . E4 are filled with accumulated charge over this time duration of 1 ms. Each node collects light for 2.5 us, one after the other in a cycle of 10 us. After the exposure time each bucket would contain 100 accumulations of its phase during the overall exposure time. This kind of sensor already existing in as a commercial product. Typically, time-of-flight sensor that is being used for 3D sensing systems for various applications, in this invention we utilize this functionality for demodulation of the returned interferometry from the sample exposure time of the sensor may have many cycles of modulation. One such sensor is Teledyne Hidra 3D, a 3-Node sensor that is having array of 640×480 pixels. Can be operated up to ~400 frames per second.

In case the phase modulation is done at the reference arm, the actual receive cycle time may slightly differ from the modulation frequency due to doppler effect. By: ƒₛ=ƒₘ+2vᵣ/λ. Where ƒₛ—is the sampling frequency, ƒₘ is the phase modulation frequency vᵣ is the OPL velocity [m/s] and λ is the central wavelength. due to the reference arm OPL change velocity due to doppler effect. E.g. for modulation of 100 kHz and OPL velocity is 1 mm/sec and wavelength=1 um. The Sampling frequency will be 102 kHz.

This method enables to obtain the full data Required for the modulation amplitudes evaluation within very short duration without being worried about sample motion during capture since the overall exposure time is quite short relative to 4 different exposures and read-out.

The example here of 4 nodes pixel type is one embodiment where we can use various number of pixel nodes starting from 2 to N where N can be any reasonable native number. In a special case of a pixel which is having only two nodes we can still evaluate the amplitudes by utilizing a small phase shift between the modulation and the nodes exposures such that the a phase between the modulated signal and the phase switching would change during the exposure to sweep along all the phases such that the phase would be correct at least part of the exposure time so the amplitude signal obtained might be smaller because of this swift however still projecting a reliable and valuable he amplitude data.

For the case of 4 node pixel the amplitude can be calculated by the following:

$${D}_{s} = - {E}_{1} + {E}_{2} + {E}_{3} - {E}_{4} \qquad ( 8 )$$

$${D}_{c} = - {E}_{1} + {E}_{2} - {E}_{3} + {E}_{4} \qquad ( 9 )$$

It was shown that:

$${A}^{2} α {D}_{s}^{2} + {D}_{c}^{2} \qquad ( 10 )$$

where A is the modulation amplitude. It was also shown that proper selection of modulation parameters can help optimize the amplitude estimation accuracy.

This description is a possible implementation of an embodiment where in the general case that as long as the phase modulator alternates the phase sufficiently for modulating the sample in the coherence window with the reference arm interference and the sensor demodulates by a sampling rate that satisfy the Nyquist theorem the conditions enable producing the desired OCT depth mapping.

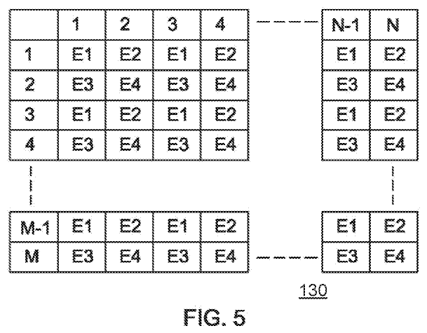

Another option to implement a multi node pixel which implement phase sensitive data accumulation over modulated signal may be done using sensor with pixel array with one node per pixel however there are several pixel types where each pixel type would integrate at different exposure window that is the pixels are different in their activation characteristics as they have different exposure timing schedulers within the sensor. Proper setting of the pixel types can obtain different phase per pixel. Enabling operation of amplitude measurement. As an example. A pixel array with 4 types of pixels may be arranged as shown in FIG. 5. The full image resolution for the 4 exposures may be obtained by means of interpolation. Like the de-mosaicking done for RGB sensor. In this method the resolution is obviously reduced relative to the 4-node pixel but might be preferred once cost and availability considerations added. The 4-pixel type case is only an example; one should note that other multi-pixel types of arrangements possible.

FIG. 5 is an example of 4-pixel type sensor arrangement. N×M array 130, E1 to E4, exposure time for each pixel type can be set to accumulate light at different timing. In this case 4 phases. Thus sensing elements E1 are activated to sense radiation at a first phase, sensing elements E2 are activated to sense radiation at a second phase, sensing elements E3 are activated to sense radiation at a third phase, and sensing elements E4 are activated to sense radiation at a fourth phase. There may be two or more phases.

### FF-TD-OCT Processing Flow.

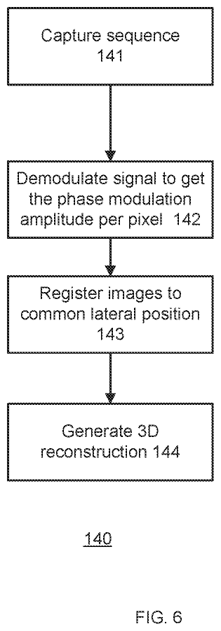

The TD-OCT is less sensitive to axial movements as the modulation is being measured directly from the signal. As discussed for the FF-SS-OCT, capture sequence (denoted 141 in FIG. 6) of images is identical, the difference is the moving optical path between samples instead of light wavelength as the SS method. A series of flashes, each flash is fast enough to eliminate lateral motion. Followed by sensor read-out time and next flashes/exposures taken until sequence end.

The process processed to step 142 of demodulating signal to get the modulation amplitude per signal—the demodulating includes calculating the modulation—for example based on E1, E2, E3 and E4. The processing flow contain a registration step 143 to align images one to the other as it would be expected that the sample would move during capture. This is illustrated at FIG. 6 that Illustrates an example of Processing flow for generating depth mapping using FF-TD-OCT

### FF-TD-OCT Enhanced Resolution.

The OCT axial resolution is determined largely by the spectral width of the light source. For TD-OCT increasing axial resolution can be done simply by increasing the bandwidth of the source. Regarding the lateral resolution. The inherent tradeoff between DOF and resolution as described above apply.

For the TD case, the tradeoff can be resolved as the optical focus distance can be synchronized with the TD reference arm path length such that the point of coherence at the sample would be within focus during scan. That is the fucus would be altered in conjunction with the optical path length to obtain high lateral resolution at the same depth plane for both. See tuneable focusing optics 45 at FIG. 2. As a result, we could leverage the OCT performance considerably. Alternatively, the focus depth can be in stepwise motion along the change of path length. Assuring the coherence window would be within DOF during scan. This way the lateral and axial resolutions are unbounded for any desired scan depth.

The focus sweep may be continuous over time in parallel to the ref arm path length change. For simplicity without big loss of performance the focus sweep may be in discrete steps of some portion of the optical DOF.

### OCT SNR Discussion.

There are many factors affecting signal quality here we briefly review major SNR influencers for the purpose of explaining benefits of our invention relative to traditional OCT.

It has been shown that an interference modulation contrast shot noise shot noise limited performance can be obtained.

### Shot Noise SNR of TD-OCT Given by:

$${SNR}_{TD - OCT} = \frac{ρ {P}_{s} {R}_{s}}{2 e B} \qquad ( 20 )$$

Where ρ is the responsivity of the detector, Pₛ is the instantaneous optical power incident on the sample, Rₛ is reflectance of the sample at the interferometry depth, B is the electrical bandwidth of the detector and e is the electronic charge.

### The SD-OCT the SNR is:

$${SNR}_{SD - OCT} = \frac{M}{2} \frac{ρ {P}_{s} [ {k}_{m} ] {R}_{s}}{2 e B} \qquad ( 21 )$$

With Pₛ[km] being the fraction of instantaneous optical power incident on the sample that corresponds to the m^{th} spectral channel of the detection channel the SS-OCT case is identical to the SD-OCT in this regard, and M is the number of detection channels (SS samples). We note that SNR_{SD-OCT} factorized by M/2. Notice that if we increase the power in the TD-OCT by the factor of M/2 both SNR's would be equivalent. In many cases, ophthalmology for example, the safety restriction would not allow for such a large power increase. In practice, the FT based methods are indeed better with regard to SNR, but in less extend given in the analysis. As the activation assumptions are different.
Using FF-OCT changes the illumination regime over the sample relative to spot or line scanning method. For spot scan—each area in the sampled region is illuminated once during scan, this means that the instantaneous power is very high for a short while. Where the FF method illuminates the entire area at the for many and relatively lower peak pulses. As a result, in the FF-OCT case the power limit is much higher thus enabling compensation overt the SD-OCT method.
Regarding SNR, due to safety regulations, especially eye safety, illumination regime of long, low peak pulse enables projecting with higher overall power. Thus FF-OCT is able to expose to higher power during the scan, this is a big advantage for FF-OCT. FF-SS-OCT is potentially the best method as it has the advantage of FF illumination regime combined with the inherent SD/SS SNR advantage.
The SD-OCT and SS-OCT Fall-Off effect causes for contrast degradation as the depth reconstruction is deeper in the sample, due to that the wavelength slices width reduces the k domain signal. This issue may be resolved as the swipe of wavelength obtained with discrete steps, so each sample gets one wavelength.
Another issue relates SD and SS-OCT is the Autocorrelation noise, describes above, it is due to the fact that the coherence length of each wavelength sample is quite wide, thus all sample internal reflectance interferes between one to another, there are methods to overcome it but with cost of price and complexity. The method FF-TD-OCT described here does not have this issue as the coherence length is low.
One more issue SD and SS-OCT is the speckle noise associated with narrow-band imaging due to the monochromatic nature of the frames this method using. In this invention FF-TD-OCT broadband light sources that features much lower speckle noise.

This invention FF-OCT advantages.

The advantages for this invention are:
Simplify optics, eliminate the need for scanning.
Enable more power projection once safety is a concern. Relative to spot or line scanning methods due to better safety conditions.
Enable concurrent usage of the same collection optics and sensor for other imaging modalities.
TD-FF-OCT case, inverse FT operation is not needed. Reduced speckle noise, no autocorrelation noise.
In-vivo usage possible. Dispersion and mechanical jitters handled easily.

All OCT methods described above, SS and TD are full-field imaging. Employing simple imaging without moving parts and sophisticated scanning optical modules, relatively low rate of illumination and data capture. These FF based OCT having cost/effectiveness/form-factor advantages over traditional OCT implementations today. Proposes, high axial resolution, diffraction limited spatial resolution and un-bounded depth range.

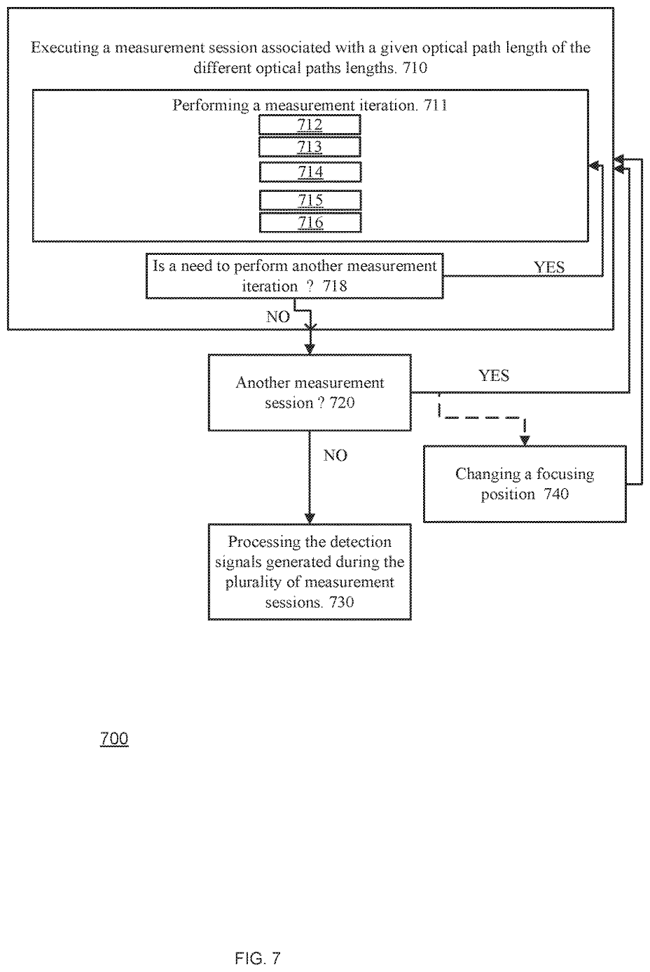

FIG. 7 illustrates an example of method 700 for method for optical coherence tomography (OCT).

Method 700 may include performing a plurality of measurement sessions. Different measurement sessions are associated with different optical paths lengths of a reference arm of an interferometer.

Method 700 may include step 710 of executing a measurement session associated with a given optical path length of the different optical paths lengths.

Step 710 includes performing measurement iterations.

Step 710 may include step 711 of performing a measurement iteration. Step 711 may include steps 712-616.

Step 712 includes illuminating a sample by radiation that passes through a sensing arm of the interferometer.

Step 713 includes phase modulating of the radiation that propagates within an arm of the interferometer, wherein the arm is selected out of the sensing arm and a reference arm, wherein the phase modulation has a modulation cycle.

Step 714 includes merging radiation that exits the reference arm with radiation from the sensing arm that returned from a sub-region of an sample to provide an interference pattern.

Step 715 includes generating signals indicative of the interference pattern by two dimensional (2D) array of sensing related elements of a sensing unit, wherein different 2D array s of sensing related elements sample the interference pattern at different detection periods within the measurement iteration. A duration of each one of the detection periods is a fraction of the modulation cycle. The duration of each one of the detection periods may be determined based on a phase modulation frequency and a rate of change of the reference arm optical paths lengths.

Step 716 includes aggregating, by the sensing unit, signals obtained by each one of the sensing related elements during the measurement iterations to provide measurement session results.

Step 711 may be followed by step 718 of checking if there is a need to perform another measurement iteration—and if so—repeating the execution of step 711.

Step 710 may be followed by step 720 of checking if there is a need to perform another measurement session—while using another optical path length of the different optical patch lengths—and if so—repeating step 710 with another value of the given optical path length.

Method 700 may also include step 730 of processing the detection signals generated during the plurality of measurement sessions.

The sensing related element may be a radiation detector. The radiation sensor may be followed by a charge accumulator.

The sensing related element may be a charge accumulator that is preceded by a radiation detector that is in communication, via a charge distribution circuit, with the charge accumulator and one or more additional charge accumulator.

The duration of a measurement session may not exceed 1 milliseconds—or may not exceed any other time threshold.

The depth of the sub-region corresponds to the given optical path length of the reference arm.

Step 730 may provide three dimensional (3D) information about the sample. The 3D information may describe the 3D structure of the sample.

Step 730 may include registering measurement session results of different sessions. The registering may include lateral position compensation or any other registration.

Step 730 may include contrast optimization.

Step 730 may include processing measurement session results from at least some of the plurality of measurement sessions to provide three dimensional information about the sample.

Step 730 may include aligning measurement session results from the at least some of the plurality of measurement sessions.

Method 700 may include step 740 of changing a focusing position of the sensing arm and preforming another plurality of measurement sessions. Thus—step 740 may be followed by step 710. Step 740 may be preceded by step 720.

A depth of the sub-region corresponds to the given optical path length of the reference arm and to the focusing position of the sensing arm.

Interference patterns obtained during the plurality of measurement sessions may be indicative of a first layer of the sample. Interference patterns obtained during the other plurality of measurement sessions may be indicative of a second layer of the sample; wherein the first layer is associated with a first depth range and the second layer is associated with a second depth range that differs from the first depth range.

Interference patterns obtained during the plurality of measurement sessions may be indicative of a first segment of the sample. Interference patterns obtained during the other plurality of measurement sessions may be indicative of a second segment of the sample. A first segment may be associated with a first height range and the second segment is associated with a second height range that differs from the first height range.

The changing of the focusing position of the sensing arm may occur along an optical path length change of the reference arm.

The OCT may be executed in vivo or in vitro. Or any other sample of interest.

It should be noted that while method 700 illustrates an example of completing a measurement session and then executing another measurement session—that the measurement iterations may be executed in any order—including jumping from one measurement iteration of a certain measurement session to another measurement of another measurement session without completing the certain measurement session.

There may be provided a device for optical coherence tomography (OCT), the device may include (a) an interferometer, (b) a radiation source, (c) a phase modulator, (d) a sensing unit that may include two dimensional (2D) array of sensing related elements; and (e) a controller that may be configured to control a performing of a plurality of measurement sessions; wherein different measurement sessions may be associated with different optical paths lengths of a reference arm of an interferometer.

During a measurement session that may be associated with a given optical path length of the different optical paths lengths, the OCT device may be configured to perform measurement iterations, wherein for each measurement iteration: (A) the interferometer may be configured to illuminate an sample by radiation that may be generated by the radiation source and passes through a sensing arm of the interferometer; (B) the phase modulator may be configured to phase modulate the radiation that propagates within an arm of the interferometer, wherein the arm may be selected out of the sensing arm and a reference arm, wherein the phase modulation has a modulation cycle; (C) the interferometer may be also configured to merge radiation that exits the reference arm with radiation from the sensing arm that returned from a sub-region of an sample to provide an interference pattern; and (E) the sensing unit may be configured to: (e1) generate signals indicative of the interference pattern by a D array of sensing related elements, wherein different D array s of sensing related elements sample the interference pattern at different detection periods within the measurement iteration; wherein a duration of each one of the detection periods may be a fraction of the modulation cycle; and (e2) aggregate signals obtained by each one of the sensing related elements during the measurement iterations to provide measurement session results.

The device may be configured to execute method 700. The device may include a processing circuit that is configured to execute step 730.

An example of a device is illustrated in FIG. 2, and examples of sensing related elements are illustrated in FIGS. 4 and 5.

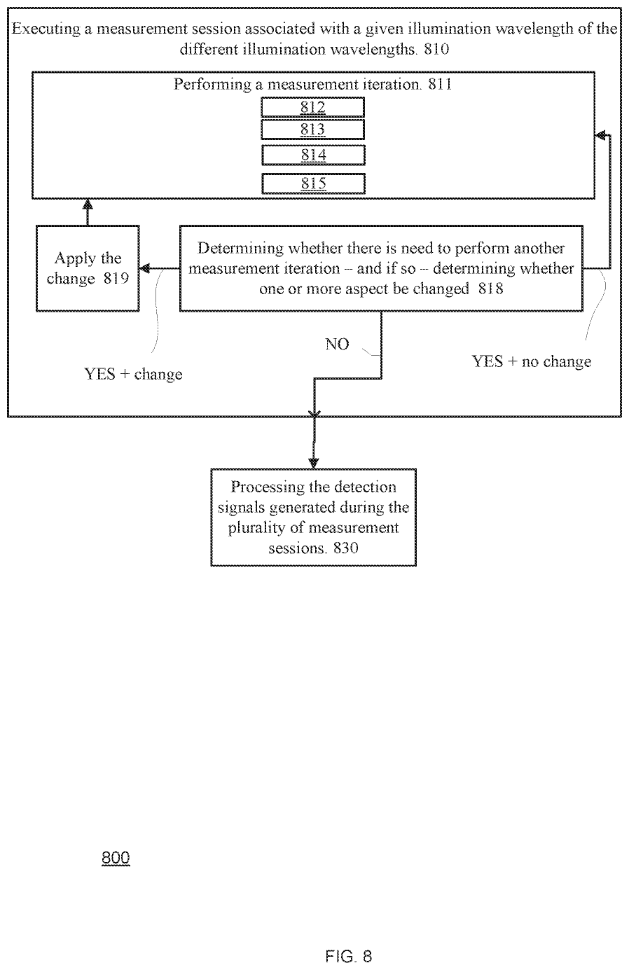

FIG. 8 illustrates an example of method 800 for method for optical coherence tomography (OCT).

Method 800 may include performing a plurality of measurement sessions. Different measurement sessions are associated with different illumination wavelengths generated by a coherent radiation source.

Method 800 may include step 810 of executing a measurement session associated with a given illumination wavelength of the different illumination wavelengths.

Step 810 includes performing multiple sets of measurement iterations. Different sets are associated with different phase shifts introduced to radiation in an arm of the interferometer, the arm is selected out of a reference arm and a sensing arm.

Step 810 may include step 811 of performing a measurement iteration. Step 811 may include steps 812-715.

Step 812 may include illuminating an sample by radiation of the given illumination wavelength, the radiation passes through a sensing arm of the interferometer.

Step 813 may include introducing the phase shift of the set in the arm of the interferometer.

Step 814 may include merging radiation that exits the reference arm with radiation from the sensing arm that returned from a sub-region of an sample to provide an interference pattern.

Step 815 may include generating signals indicative of the interference pattern by a two dimensional (2D) array of sensing related elements out of different 2D array s of sensing related elements that belong to a sensing unit, the 2D array is associated with the phase shift of the set.

Step 811 may be followed by step 818 of how to proceed.

Step 818 may include determining if there is a need to perform another measurement iteration.

Step 818 may include determining whether to change any aspect related to the next measurement iteration—for example is there a need to change a phase shift and/or whether there is need to change a focusing position of the sensing arm.

Step 818 may be followed by step 811 if there is a need to perform another measurement iteration without changing an aspect.

Step 818 may be followed by step 819 of changing one or more aspect (phase shift and/or focusing position) and jumping to step 811.

Step 818 may follow by ending the method when no more measurement iteration is required.

Method 800 may also include step 830 of processing the detection signals generated during the plurality of measurement sessions.

Step 830 may include processing measurement results of the plurality of measurement sessions to provide three dimensional (3D) information about the sample.

Step 830 may include inverse Fourier transform to generate depth related information of the sample.

Step 830 may include registering measurement session results of different sessions. The registering may include lateral position compensation or any other registration.

Step 830 may include contrast optimization.

Step 830 may include processing measurement session results from at least some of the plurality of measurement sessions to provide three dimensional information about the sample.

Step 830 may include aligning measurement session results from the at least some of the plurality of measurement sessions.

It should be noted that while method 800 illustrates an example of completing a measurement session and then executing another measurement session—that the measurement iterations may be executed in any order—including jumping from one measurement iteration of a certain measurement session to another measurement of another measurement session without completing the certain measurement session.

There may be provided a device for optical coherence tomography (OCT), the device may include (a) an interferometer, (b) a coherent radiation source, (c) a phase shifter, (d) a processing circuit, (e) a sensing unit that comprises two dimensional (2D) array of sensing related elements; wherein different 2D array s are associated with different phase shifts; and (f) a controller that is configured to control a performing a plurality of measurement sessions. Different measurement sessions are associated with different illumination wavelengths generated by the coherent radiation source.

During a measurement session that is associated with a given illumination wavelength of the different illumination wavelength the device is configured to perform different sets of measurement iterations, wherein the different sets differ from each other by a phase shift introduced to radiation in an arm of the interferometer and by the phase shifter, the arm is selected out of a reference arm and a sensing arm.

During a measurement iteration of a set of measurement iteration that is associated with a given phase shift: (A) the interferometer is configured to illuminate an sample by radiation of the given illumination wavelength, the radiation passes through a sensing arm of the interferometer; (B) the phase shifter is configured to introduce the given phase shift of the set in the arm of the interferometer; (C) the interferometer is configured to merge radiation that exits the reference arm with radiation from the sensing arm that returned from a sub-region of an sample to provide an interference pattern; (D) a 2D sensing array that is associated with the given phase shift is configured to generate signals indicative of the interference pattern.

The processing circuit may be configured to process measurement results of the plurality of measurement sessions to provide three dimensional (3D) information about the sample.

An example of a device is illustrated in FIG. 2, and examples of sensing related elements are illustrated in FIGS. 4 and 5.

### Multiple Operational Modes

### Combining FF-OCT+Imaging

The FF-OCT, regardless of if its TD, SS as described in this invention, utilizes 2D imaging optics from the sample to 2D sensor area. The sensor may be low-cost CMOS, (multi-node at TD case). Given this design one can combine various other modes of imaging using the same detector array and optics. A great advantage for cost reduction and form factor.

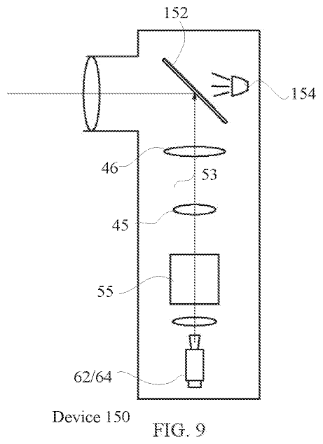

Referring to Error! Reference source not found. FIG. 9. The device 150 differs from device 30 of FIG. 2 by replacing folding mirror 53 by a second beam splitter 152 and adding a second light source 154. The device 150 may include additional elements—as shown in the front view of FIG. 2.

For all the non-OCT modes the, interferometer reference arm may disrupt the imaging path, hence should be blocked from reaching the sensor. An optical dumper may be inserted in the reference path. Or replacing the first beam splitter with a mirror.

### RGB Imaging+OCT

The OCT typically uses the IR wavelength it can be at the range of 800-1300 nm, we can also project visible broadband illuminating (400-700 nm) at light source 154 and review the obtained RGB image a consumer-based CMOS sensor with Color Filter Array (CFA). Typically named RGB sensor or equivalently use alternating light Red, Green and Blue to get RGB images of the sample.

If the device is used for fluorescence, then a filter may be added to prevent excitation wavelength radiation from reaching the sensor in the imaging path. The beam splitter may be a dichroic beam splitter that passes the excitation wavelength and reflects the fluorescence wavelength. The second light source may be various options such as white light, broadband VIS+NIR or optionally narrow band of any desired wavelength. The light returning from the sample will partially be reflected from the beam-splitter to the 2D sensor direction, as it's the same imaging optics, the sensor will generate an image of the sample. The sensor may be spectrally sensitive such as RGB sensor providing colored image and the spectral content of the sample may be analyzed.

### Spectroscopy

Another modality of use is spectroscopy. Using A light source 154 that is the exit of combined plurality of wavelengths. A set of LED's each at different wavelength are all directed into optical fiber, each wavelength can be activated separately one after the other, images of the sample can be captured per wavelength. From the plurality of images an accurate spectroscopy analysis can be done. This light source can also be obtained with broadband illumination source and a plurality of bandpass filters each tuned to different wavelength. Both these methods can provide spectral data for analysis. Registration between images of different wavelength can further be used to better align features as the fixation jitters may reduce image quality during scan. The capture processing flow given at FIG. 6 may be applied here.

### Polarization.

Controlling the illumination polarization and/or collection path polarization can enable distinguishing polarization specific objects in the sample.

### Fluorescence

Fluorescence imaging can be obtained in the device, referring to FIG. 9, adding a second light source 154 via a second beam splitter, 152 that may be a dichroic beam splitter, where this light source emits light at certain excitation wavelength. And by insertion of an optical spectral filter in the receive path of the optical path between the beam-splitter and the 2D sensor this filter designed to block the excitation wavelength and to pass the fluorescence wavelength, it can be bandpass filter, high pass or low pass. Applying illumination with one wavelength and collection with other wavelength range, the use of optical filter blocks the illumination light and collected only the desired wavelength range, obtaining fluorescence image at the 2D sensor.

When a device is used for OCT and fluorescence—thus the device includes an interferometer—then when operating in a fluorescence mode the reference arm of the interferometer is not used. The 2D sensor may also have a Color Filter Array (CFA), to provide spectral information about the fluorescence wavelength and subtract residual noise from the illumination wavelength. Example: suppose the light source wavelength λi=450 nm and the fluorescence emission may be 500-800 nm. Illumination pulse duration can be 1-2 ns and using 1:1000 cut off spectral filter, there may be a residual back reflected power that can compete with fluorescence power. Using the sensor CFA can help us distinguish between the source signal and fluorescence.

### Fluorescence Lifetime FLIO and FLIM

Fluorescence Lifetime Imaging Microscopy (FLIM) and Fluorescence Lifetime Imaging Ophthalmoscopy (FLIO) are imaging method where the florescence of the sample decay time is measured. In this context we regard FLIM as the general case where FLIO and other florescence lifetime measurements are specific cases. Biological materials exhibit fluorescence activity not only characterized by the wavelength and energy of emission, but the fluorescence emission decay time is indicative as well.

The fluorescence lifetime is the average amount of time a fluorophore remains in the excited state following excitation. In recent years there has been huge advancement in this field where dual lifetime composition became relevant for the detection of biological activity, one such method called Förster Resonance Energy Transfer (FRET). The method measures biological activities between donor and acceptor molecules and can give indication of the presence of acceptor molecules near the donor. The donor molecule is typically a fluorescence type with a known typical decay lifetime. But with the presence of acceptor molecule some of the pumped electrons in the higher energy level pass the acceptor at a different rate instead to produce fluorescent illumination. As a result, the fluorescent decay time exhibits 2 or more decay coefficients. In the general case of dual lifetimes, the fluorescent signal can be described as:

$$Fl ( t ) = {I}_{0} {e}^{- t / {τ}_{I}} + {J}_{0} {e}^{- t / {τ}_{J}}$$

That is a superposition of 2 exponential decay times with I₀ and J₀ is their initial strength and τ_{I} and τ_{J} their decay lifetime. In many cases the lifetimes τ_{I} and τ_{J} are known where the strength is the interesting feature. Assuming I₀ and τ_{I} are the donor inherent property with the absence of the acceptor molecule, the measurement of J₀ and τ_{J} will sign for the acceptor molecular concentration and/or biological activity. This description is only basic explanation of the importance of the dual lifetime measurement.

There are two basic methods for FLIM measurement, frequency domain and time domain. The time domain typically referred to as Time-Correlated Single Photon Counting (TCSPC) is commonly being used for FLIM as it provides good measurement, but in some cases the frequency domain is being used. Florescence light energy is typically much less than the illumination power by 1-3 orders of magnitude, thus one needs a sensitive collection optics and sensor for accurate and reliable reading. Some FLIM methods use single-photon avalanche diode (SPAD) sensor array combined with photon arrival timing circuits, having high sensitivity but typically at low spatial resolution relative to consumer-based CMOS sensor. A TCSPC method was proposed to provide a FLIM estimate that is based on heterodyne slice-based detection of the signal using fast gated camera.

According to an embodiment, a Time of Flight (TOF) sensor can be utilized for the FLIM measurement, especially the usage of multi-node pixel sensor described above. The advantage of TOF sensor is its price as it's being used for consumer market in many applications. Also, its output data rate is equivalent to regular consumer-based sensors. A typically TOF sensor modulation rate ranges 80-300 MHz, that is the exposure timing of the sensor can be switched on/off at durations smaller than few ns.

FIG. 11 includes timing diagram 160 that illustrates short and long lifetimes (curves 161 and 162 respectively) for infinitesimal short excitation light pulse. The overall fluorescent emission (curve 163) is a superposition of the 2 separate responses.

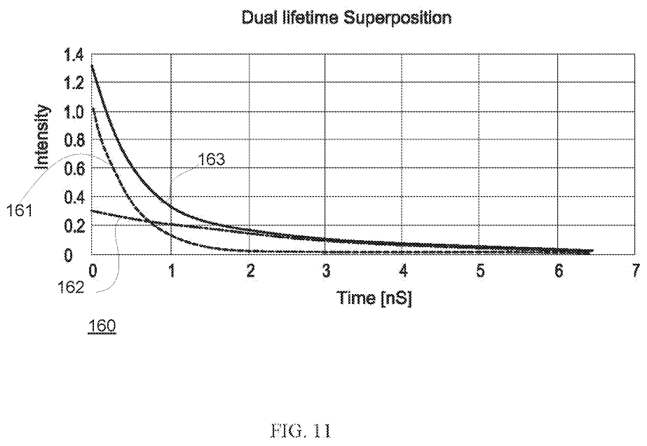

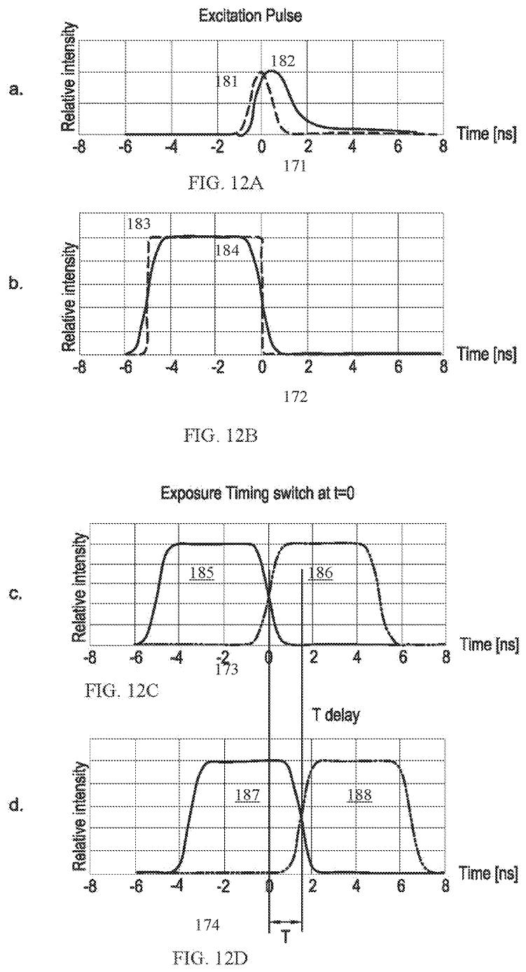

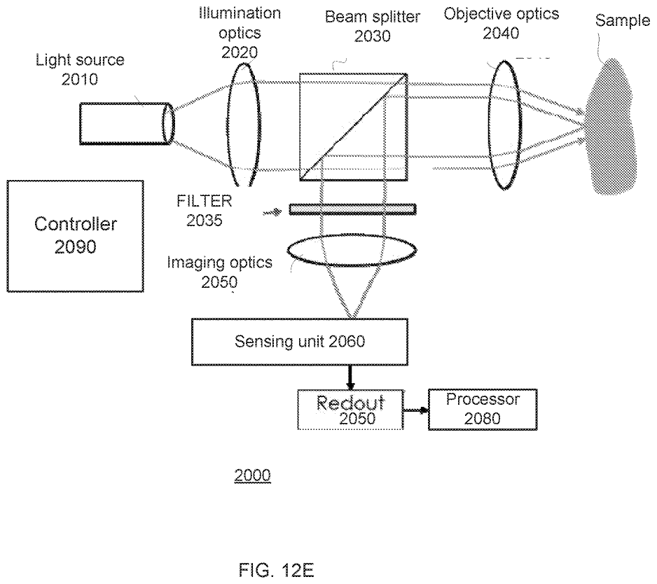

FIGS. 12A-12D illustrate example of timing diagram of the fluorescence light (FL) emission. Timing diagram 171 of FIG. 12A illustrates the excitation emission pulse 181 and the fluorescence response 182. Timing diagram 172 of FIG. 12B illustrates the sensor exposure timing) ideal 183 and actual 184), synchronized to start prior to FL pulse and ending at t=0. Exposure of the pixel photodiode is an analog process with typical rise-time and fall-time that the rise or fall time duration can be at the order of the FL signal. Timing diagram 173 of FIG. 12C illustrates a 2-node pixel sensor where the 2 exposures 185 and 186 are sequence one after the other. Timing diagram 174 of FIG. 12D illustrates a 2-node pixel sensor with delayed phase time=T.

Denote Fₗ(t) as the fluorescence intrinsic response to ideal excitation pulse emission of the inspected material. And A(t) is the actual excitation temporal profile. Than:

$$S ( t ) = {F}_{l} ( t ) \text{?} A ( t )$$

Where S(t) is Fl temporal emission, and ⊗ denote the convolution operation. The sensor exposure time can be configured such that its exposure time ends at time t=0 ns. The exposure starts long before the excitation pulse starts and ends at t=0 ns as noted, the exposure time ΔT can be set to long duration. If the sensor exposure is an ideal top-hat than the collected charge in the sensor pixel would represent the integration cross section with the S(t) signal. But since the exposure is also an analogue signal the pixel readout would be:

$$P ( T = 0 ) = {\int }_{- Δτ}^{0} S ( t ) \cdot α ( r ) dt$$

Where α(t) is the analog representation of the exposure responsivity in time. The phase of exposure timing can be configured to any desired timing to obtain phase T dependent charge integration:

$$P ( T ) = {\int }_{t 1}^{t 2} S ( i ) \cdot α ( t - T ) dt = {\int }_{- ∞}^{∞} S ( t ) \cdot α ( t - T ) dt = S ( t ) \text{?} α ( t - T ) ;$$

Denote t₁ as the excitation pulse start and t₂ as the time S(t) decays to noise level. The exposure time configured to ΔT>(t₂−t₁). The P(T) effect is integrative over S(t) from excitation pulse start till the exposure end or S(t) reduces to noise level. Obtaining value of P(T) for T=t₁ to t₂ done by incrementing the phase T that can be configured for a sequence of N exposure steps {Tᵢ}; i=1 . . . N, Where the Tᵢ Increments can be (t₂−t₁)/(N−1). Note that the {Tᵢ}can be spaced in non-linear way for optimization purposes. For {Tᵢ}spacing that satisfy Nyquist sampling theorem the pixel reading values set {P(Tᵢ)}can be interpolated to continuous time base, resampled & manipulated for the purpose. Remembering that what we look for is the fluorescence decay time of Fₗ(t) the pixel signal over the sequence of phase sweep given by:

$$P ( T ) = {F}_{l} ( t ) \text{?} A ( t ) \text{?} α ( t - T ) ;$$

$$And define \text{:} I ( T ) = A ( t ) \text{?} α ( t - T )$$

Where I(T) is the instrument response for the given excitation pulse and phase delay T. Finaly we can calculate the desires fluorescence signal Fₗ(t) by Fₗ(t)=deconvolution(I(T), P(t)).

The usage of a TOF sensor allow for accumulations of many pulses for each phase capture Tᵢ this is very significant for having adequate SNR for the detection signal. As the series of exposure may be in the order of 1e3 to 1e5, depending on the system sensitivity and required throughput.

Increasing accumulation count increase the photon collection and helping overcoming dark noise, shot noise and other random noise sources.

A numerical example for such a sequence: the typical lifetime of the decay can be 0.5 to 5 ns. Also assume the excitation pulse duration [−1 ns:1 ns], so the overall S(t) will span at the range [t₂, t₁]=>[−1 ns:6 ns] We can set the phase Ti steps between −1 nm to 6 nm with steps of 0.1 ns using a programable and accurate delay line. Next we can set the sensor exposure time ΔT=10 ns that satisfies (ΔT>(t₂−t₁)). Each capture sequence can accumulate over N=5000, pulse/accumulation sequences. After each capture the pixel charge will accumulate over 5000 steps allowing for collecting high count of photons, thus no SPAD needed. The pulse rate duration should be long enough to avoid Fl tail accumulation, for pulse rate of 50 ns and 5000 pulses the overall sequence duration is 250 us=0.25 ms. Following the sequence a read out of pixel charge process done to get P(Ti) for i=1, The process of accumulation and read out repeats for i=2 to i=71 (−1 ns to 6 ns with 0.1 ns steps). Modern sensors read-out duration may be ~2-5 ms. Thus the overall capture sequence of the 71 frames is 71*(0.25 ms+5 ms)=372.75 ms that is the whole capture sequence duration is <1 sec.

Further improvement can be gained with the use of 2 node sensor using a second exposure time for the second node, noted as β(t). Starting right after α(t) for the same exposure duration, β(t) starts the exposure at T for duration of ΔT. Similarly the end of its exposure should be later that t₂ for all phases. See illustration at FIG. 12D. Define

$${α}_{n} ( t ) = \frac{α ( t )}{α ( t ) + β ( t )}$$

The new exposure time αₙ(t) is similar to its origin α in the relevant region. However since the denominator [α(t)+β(t)] collects the entire pulse energy it normalize the signal for RIN noise sources such as pulse to pulse variations and drifts. Actually, the complementary assumption of the sum [α(t)+β(t)] may not be guaranteed, it depends on sensor internal realization of node switching. Some such sensors may exhibit such behavior or sufficiently complementary as the photo-electrons in the photodiode well are collected to one of the nodes.

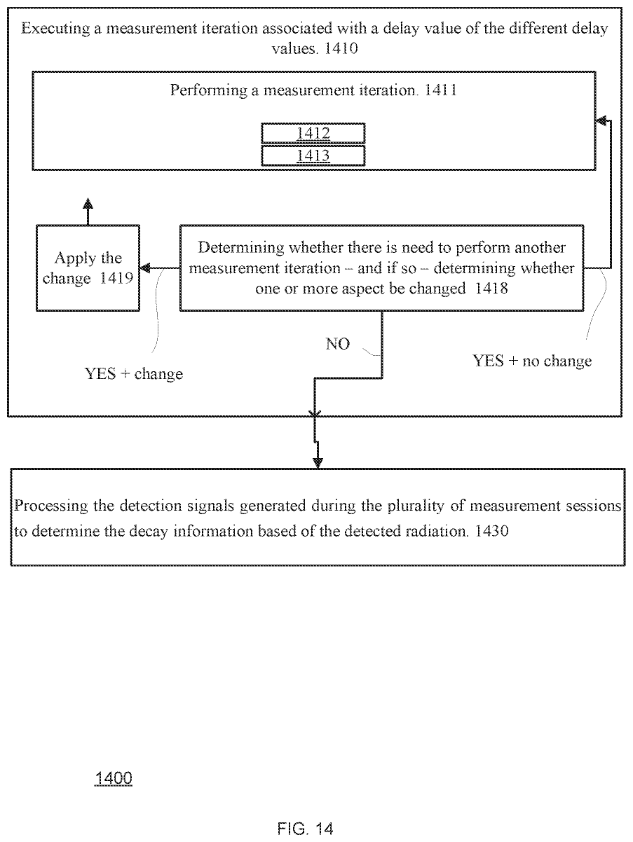

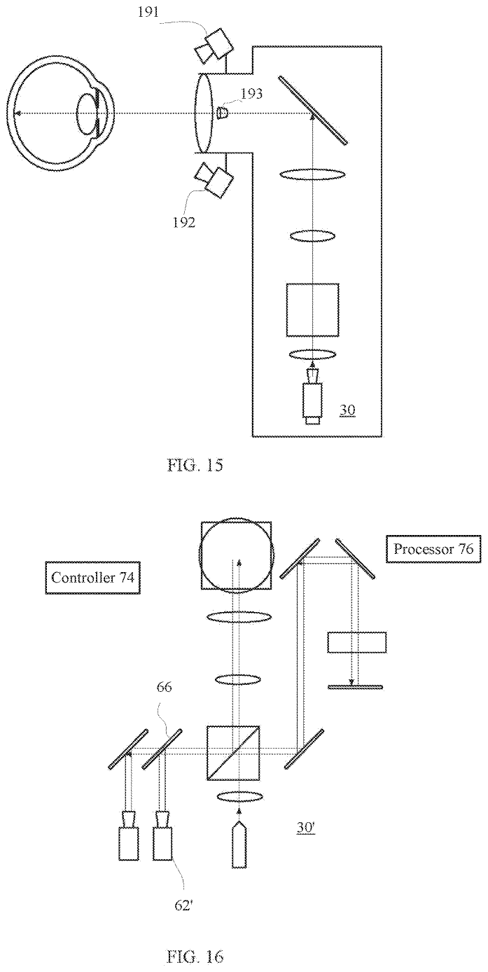

FIG. 14 illustrates an example of method 1400 for determining fluorescence decay information. Method 1400 may include performing a plurality of measurement sessions; wherein different measurement sessions are associated with different delay values.

Method 1400 may start by step 1410 of executing a measurement iteration associated with a delay value of the different delay values.

A delay value may be positive or negative.

Step 1410 may include steps 1411, 1412 and 1413.

Step 1411 may include illuminating a region of a sample with radiation pulses that result is a generation of fluorescence pulses. The radiation pulse forms a two dimensional spot (2D) on the region.

Step 1412 may include detecting radiation, by a 2D detector of a sensing unit, during detection windows that start at the given delay value from starts of the radiation pulses. Each detection window has a duration that (i) exceeds a duration of the fluorescence pulse, and (ii) does not exceed a time difference between adjacent radiation pulses.

Step 1413 may include aggregating, by the sensing unit, detection signals obtained during the detection windows that start at the given delay value from starts of the radiation pulses.

Step 1411 may be followed by step 1418 of how to proceed.

Step 1418 may include determining if there is a need to perform another measurement iteration.

Step 1418 may include determining whether to change any aspect related to the next measurement iteration—for example is there a need to change a phase shift and/or whether there is need to change a focusing position of the sensing arm.

Step 1418 may be followed by step 1411 if there is a need to perform another measurement iteration without changing an aspect.

Step 1418 may be followed by step 1419 of changing one or more aspect (phase shift and/or focusing position) and jumping to step 1411.

Step 1418 may follow by ending the method when no more measurement iteration is required.

Step 1410 may be followed by step 1430 of processing the detection signals generated during the plurality of measurement sessions to determine the decay information based of the detected radiation.

As indicated above—one measurement iteration may be executed after the other—even regardless of the measurement session associated with the measurement iteration.

The plurality of measurement sessions may include pairs of measurement sessions, at least one pair of measurement sessions comprises a measurement session of a positive delay value and a measurement session of a negative delay value.

The at least one pair of measurement sessions may include a measurement session of a positive delay value and a measurement session of a negative delay value.

The at least one pair of measurement sessions may include a fluorescence pulse rise measurement session and a fluorescence pulse fall measurement session.

The fluorescence pulse rise measurement session may include fluorescence pulse rise detection windows, wherein the fluorescence pulse fall measurement session may include fluorescence pulse fall detection windows, wherein the fluorescence pulse rise detection windows and the fluorescence pulse fall detection windows are interleaved.

For the at least one pair of measurement sessions, step 1413 may include aggregating, by a first aggregation element, detection signals obtained detection windows of a first measurement session of the pair, and aggregating, by a second aggregation element, detection signals obtained detection windows of a second measurement session of the pair.

Step 1430 may include applying a deconvolution operation.

The decay information may be indicative of two decay time constants.

There may be provided a device for determining fluorescence decay information, the device may include, a radiation source, a processing circuit, a sensing unit that may include two dimensional (2D) array of sensing related elements; and a controller that may be configured to control a performing a plurality of measurement sessions. Different measurement sessions are associated with different delay values.

During a measurement session that may be associated with a given delay value: (a) the interferometer may be configured to illuminate a region of a sample with radiation pulses from the radiation source that result may be a generation of fluorescence pulses; wherein a radiation pulse forms a two dimensional spot (2D) on the region; (b) a 2D detector of the sensing unit may be configured to detect radiation during detection windows that start at the given delay value from starts of the radiation pulses; wherein each detection window has a duration that (i) exceeds a duration of the fluorescence pulse, and (ii) does not exceed a time difference between adjacent radiation pulses; (c) the sensing unit may be configured to aggregate detection signals obtained during the detection windows that start at the given delay value from starts of the radiation pulses; and (d) the processing circuit may be configured to determine the decay information based of the detected radiation.

At least one of the delay values may be positive and at least one of the delay values may be negative.

The plurality of measurement sessions may include pairs of measurement sessions, at least one pair of measurement sessions may include a measurement session of a positive delay value and a measurement session of a negative delay value.

The plurality of measurement sessions may include pairs of measurement sessions.

The at least one pair of measurement sessions may include a measurement session of a positive delay value and a measurement session of a negative delay value.

The at least one pair of measurement sessions may include a fluorescence pulse rise measurement session and a fluorescence pulse fall measurement session.

The fluorescence pulse rise measurement session may include fluorescence pulse rise detection windows, wherein the fluorescence pulse fall measurement session may include fluorescence pulse fall detection windows, wherein the fluorescence pulse rise detection windows and the fluorescence pulse fall detection windows are interleaved.

For the at least one pair of measurement sessions a first aggregation element of the sensing unit may be configured to aggregate detection signals obtained detection windows of a first measurement session of the pair, and wherein a second aggregation element of the sensing unit may be configured to aggregate detection signals obtained detection windows of a second measurement session of the pair.

The processing circuit may be configured to determine the decay information by applying a deconvolution operation.

The decay information may be indicative of two decay time constants.

FIG. 12E illustrates an example of a device 2000 for determining fluorescence decay information. The device may include a radiation source 2010 such as a light source that is followed by illumination optics 2020, beam splitter 2030 and objective optics 2040 to provide an illumination path that illuminates a 2D area of the sample.

The sample may emit (i) fluorescence radiation and (ii) additional radiation at the frequency of the illuminating radiation.

The fluorescence radiation propagates through the objective optics 2040 to the beam splitter 2030 and is directed through imaging optics 2050 to the sensing unit 2060. Signals generated by the sensing unit 2060 are read by readout circuit 2070 to provide detection signals that are accessible to the processor 2080. The device is controlled by controller 2090.

The additional radiation (at the frequency of the illuminating radiation) propagates through the objective optics and is either blocked by the beam splitter (which may be a dichroic beam splitter) may be directed by the beam splitter to a filter 2035 that blocks the additional radiation. Blocked means that the additional radiation does not reach the sensing unit.

The device may be without a interferometer.

Device 2000 may be configured to execute method 1400.

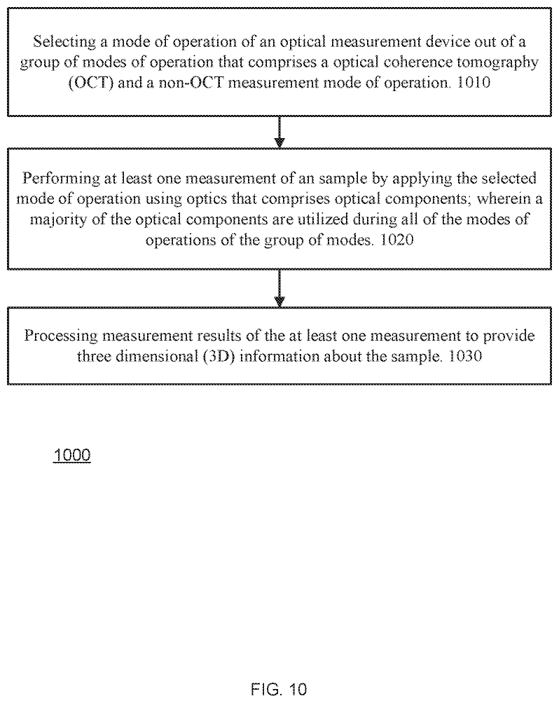

FIG. 10 illustrates an example of method 1000 for multimode measurements.

Method 1000 may start by step 1010 of selecting a mode of operation of an optical measurement device out of a group of modes of operation that comprises an optical coherence tomography (OCT) and a non-OCT measurement mode of operation.

Step 1010 may be followed by step 1020 of performing at least one measurement of a sample by applying the selected mode of operation using optics that comprises optical components; wherein a sensing unit and at least a majority of optical components of an imaging path are utilized during all of the modes of operations of the group of modes.

Step 1020 may be followed by step 1030 of processing measurement results of the at least one measurement to provide information about the sample.

The information may include three dimensional (3D) information about the sample, delay information, and any information from any model.

Method 1000 may include fusing all these modalities data for diagnostics/feature detection and/or may determining information about the sample using information obtained from measurement obtained by different modes of operation and/or different modalities.

The information about the sample may include at least some of the following:
1. 3D OCT mapping
2. Fluorescence image.
3. Fluorescence Decay information.
4. RGB images.
5. A set of spectroscopic images.

The non-OCT measurement mode of operation may be selected from a Fluorescence Lifetime Imaging Microscopy measurement mode, spectroscopy, Fluorescence mode of operation, and the like.

Examples of OCT measurements that may be applied in method 1000 are illustrated in FIGS. 7 and 8.

There may be provided a device for multimode measurements, the device may include optics that comprises optical components; and a controller that is configured to: select a mode of operation of an optical measurement device out of a group of modes of operation that comprises optical coherence tomography (OCT), and a non-OCT measurement mode of operation; and control a performing of at least one measurement of an sample by applying the selected mode of operation using the optics; wherein a sensing unit and at least a majority of optical components of an imaging path are utilized during all of the modes of operations of the group of modes.

The device may include a processing circuit that is configured to process measurement results of the at least one measurement to provide three dimensional (3D) information about the sample.

The device may execute method 1000. Steps of method 1000 may include executing any method of methods 700 and 800.

Examples of a device is illustrated in FIGS. 9 and 16. The device may include the elements of the device of FIG. 2 and additional elements. Examples of sensing related elements are illustrated in FIGS. 4 and 5.

Device mounted on Slit-Lamp Slit-Lamp (SL) station is a common tool for ophthalmologists to examine the patient eye.

Typically, it contains a white light source and a slit mirror to reflect the light source to illuminate the eye posterior or anterior. With imaging optics, The Slit-Lamp device is very common tool that is present at ophthalmology clinics and optometrists.

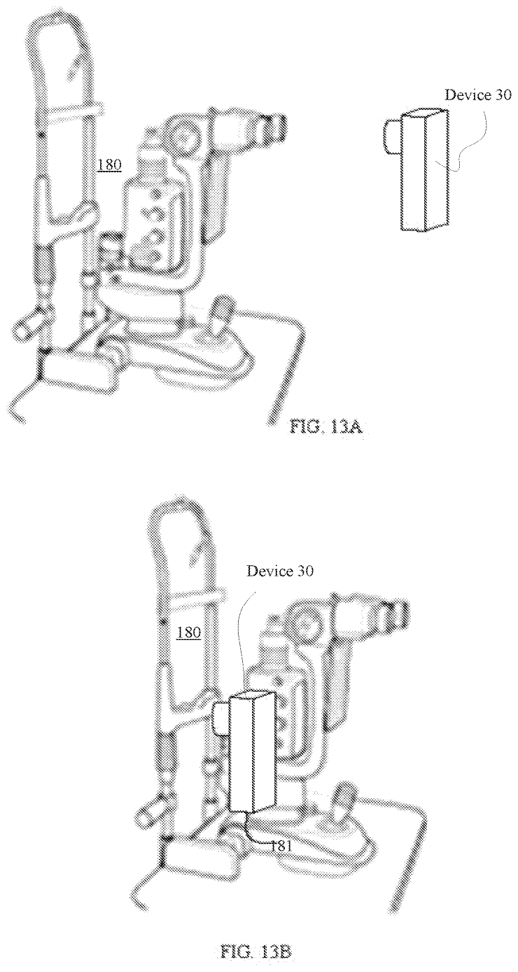

This SL station have a fixed chin rest fixture where the patient places the head and a 3D motion stage to align the optics to the patient's eye. In this invention we employ this capability by mounting the device to the SL while using its chin-rest fixture and 3D motion stage to align it to the eye. This way the ophthalmologist can combine the device of this invention multi-modal inspection and other types of tests one after the other, saving time and space, where tests like OCT, fundus imaging, fluorescence, internal eye pressure and others can be done. FIG. 13A illustrates the invention device 30 and the SL station 180 while FIG. 13B illustrates the OCT devices that is attached to the SL station.

### Auto Align Method

FIGS. 13A and 13B illustrates the device before being mounted and after being mounted on exemplar SL station. A control line 181 illustrates a connection between the device and the SL for auto align method where the device captures images with its main imaging path and dedicated small cameras to review the eye position and provide directions to the 3D motion stage for alignment.

FIG. 15 Illustrates an embodiment of eye exam device 30 with two small alignment cameras 191 and 192 and light source/s 193.

The auto-align system comprises 2 or more small camera modules at the sides of the optical path to capture the eye and use a method of stereovision to detect and align the main optical path in front of the eye. Also, small illumination modules, can be LEDs, are illuminating the eye to help the alignment camera have good quality imaging of the eye.

It should be noted the description here of eye review device is exemplar, where the tested sample can be anything with an interest to be measured with the device.

The device computer captures images from the alignment cameras, detect the eye or the desired sample object and passes commands to the SL stage for proper alignment. For that purpose, it's well understood the a calibration process needed for the stereo-vision to work properly to accurately make the alignment.

### Collection Path Split to First and Second Camera:

It may be for some cases the different imaging modalities will require usage of different sensor e.g. one for OCT and the other for spectral imaging. The receive optical path may be split via using bean splitter such that the entire imaging optics will be shared among both sensors. This way size and cost of the device maintains low.

FIG. 16 illustrates an example of a device 30′ that differs from device 30 of FIG. 2 by having an additional camera 62′ and having another beam splitter 66 that splits the light between camera 62 and additional camera 62′.

### Neurodegeneration Detection

Increasing evidence showing that the eye retina may be helpful for neural condition biosensing and provide bioindication of neuronal degradation of the brain. Dementia diseases like Alzheimer Disease (AD) exhibit neuronal degradation long before clinical indication is evident, currently MRI, PET and Cerebrospinal fluid (CSF) tests are being used for neuropathology detection, but these methods are having sensitivity and specificity issues. There is no accurate biosensing available at present days for the early signs of these diseases.

Having a reliable detection non-invasive and commonly available tests to the Neurologist once they examine the patients have a huge potential. Another aspect of need for neurodegeneration detection is treatments of new drugs administration in the field. These new drugs need a reliable tracking over the progress of the disease related features. In the process of approval, the drug companies need to perform trials over large quantity of people and since the nature of the disease progress measured in years the trial time is long, during this time the people participating in the trial need to be routinely monitored. Thus, a need for widespread easy to use testing device and system is needed for tracking over disease progress, disease related features such as the retina neural layer thickness and other such measures can be registered over time and provide critical data for effect of the drug treatment.

The neurodegeneration expression of dementia in the brain activity may be expressed in different measures like Electroencephalogram (EEG), magnetoencephalography (MEG), or Functional Near-Infrared Spectroscopy (FNIRS) see [12], that sensing the brain activity over time. Patients are given a task to perform and the brain activity during the task is measured. Both EEG and FNIRS are simple, easy to use and can be widespread easily.

A wide point of view on this subject matter is showing many methodologies that give indication for neurodegeneration but there is no complete and reliable solution for bio-indication that is also low-cost, simple and widespread.

There is provided a solution for bio-indication that is also low-cost, simple and widespread—and capable of collecting data from patients using variety of sensing modalities. Each modality may have a Sensitivity and Specificity measures that might not be sufficient stand alone, however the combination of multiple measures may provide complementary measures for obtaining a reliable result of Sensitivity and Specificity.

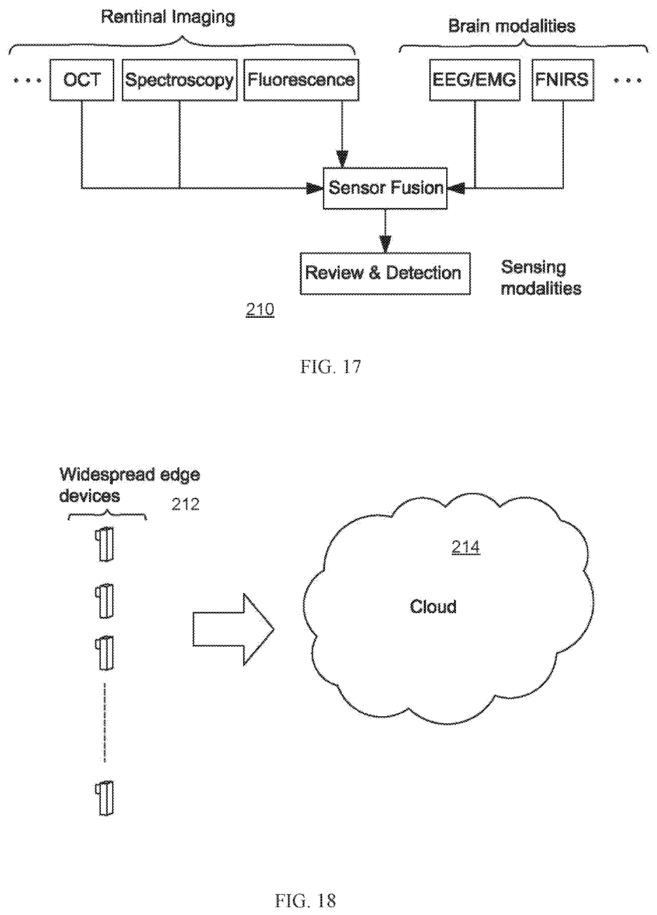

FIG. 17 illustrates a device 210 for neurodegeneration detection and tracking combining retinal imaging modalities such as OCT and fluorescence with brain sensing modalities all the modalities have been shown in the literature as having an indication to serve as bio-indication for Dementia such as AD before clinical indication but not sufficient as stand-alone test. It's the understanding of collaboration of many modalities may get the desired results.

Multiple sensing modalities data combined together in the Sensor Fusion block for coordination of temporal registration between modalities, also for regional information in the retina modalities data. Giving solid and wide basis for further pattern detection for detection and review by experts.

The capture of data my accompanied by administration of bio-indicative agents to the patients prior to the test. Such as Curcumin. Curcumin, or diferuloylmethane, exhibits an affinity for Amyloid Beta (Aβ) aggregates. Curcumin is a food constituent and considered to be safe for administration. Curcumin and its conjugates bind to β-pleated sheets of Aβ, as well as its oligomers, fibrils and plaques. More recently, the fluorescence imaging of amyloid deposits using curcumin-Aβ interactions has been applied to image Aβ deposits in the retina. Where Aβ accumulation in the brain is one of AD signs.

### Widespread Distributed Data Collection and Cloud Usages.

One of the reasons there is no widespread reliable bio-indication of neurodegeneration process in the brain and early bio-indication for dementia is the lack of measurements of people over time when the neurodegeneration process progresses since the disease diagnosis done long after process starts. There is provided a method for constructing an infrastructure for data collecting at large scale that will provide means for diagnosis development, monitoring over time, data sharing, review treatment monitoring and AI based indication alarms for possible Dementia disease.

FIG. 18 illustrates a widespread data collection 212 from edge devices into clude. Providing means for storage treatment proofing and diagnosis development. The figure illustrates the structure of widespread network of data collection involving the distribution of edge devices in clinics around the world. These edge devices, as described above would capture retina and other brain related information. All described above, thus no need to repeat here. The data collection may be implemented in a cloud service 214 such as AWS. This vast collection of data will be used for many services such safe and secured storage, sharing with experts, routine data collection from patient enabling progress tracking of disease related features. Diagnosis services offering. And new diagnosis development once the data collection quantity will be large and enable Machine Learning (ML) method such as AI based learning tools to offer reliable diagnosis. Furthermore, over time it would be expected that some of the monitored people will get eye disease or dementia, the fact that the persons data over time that precedes the diagnostics of the disease will further enhance the ML capability to provide reliable diagnostics.

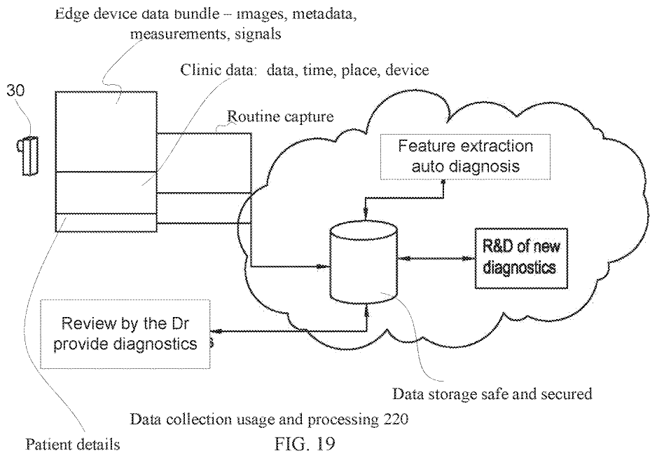

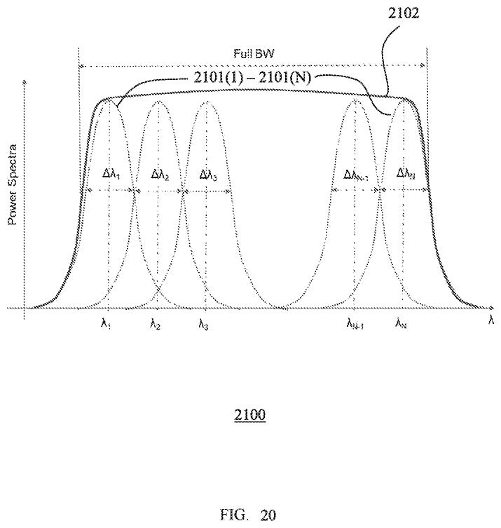

FIG. 19 illustrates data management 220 in the database. That may be a cloud service. Collected data from edge devices containing retina imaging and other sensing modalities is uploaded into storage. The data should incorporate details of the tested person such as age, gender and other relevant information including its health history. Also, the time and place, the capturing device and any other relevant information.

The collected data is being saved in a repository that is safe and secured according to the relevant standards in the health field to ensure its safety and security. The storage management should allow for authorized persons such as the person's Dr' or an expert in the field to review and make his diagnostics. The Dr' may review all relevant data including past data over time, thus the Dr' may update the diagnostics back to the repository.

Also, the storage may enable an analysis tool to extract additional features from the data such as the fluorescence level and area captured in the retina imaging, or how the OCT imaging is indicative to AD.

The extracted features and the raw data may be available to diagnostic program to inspect the persons data and features and provide its suggested diagnostic back to the storage and alarm the Dr' or its results. Note this tool do not be exposed to person's private data. Thus, no worry of patients privacy inference. As a result, these diagnostic tools may be offered by third party such as a private company or a health institute, these third-party diagnostics providers will need authorization and should follow standards of safety and security as well such that the information will not leek to not authorized users.

The aggregated data collected over time of many people would become the basic building blocks for further innovative ML based new diagnostics that would be developed as the repository grow in time. In the beginning the diagnosis will provide features measurements and some likelihood of diseases of the eye or dementia, but in time, as the data will include true diagnostics from clinical trials or other diagnostics tools such as PET the ML-based diagnostics would improve sensitivity and specificity up the high confidence level of early onset bio-indication.

One more service the system can provide is monitoring over the performance of new drugs. People participating in a trial of new drugs for dementia such as AD or an eye disease are monitored repeatedly with this system, the data storage is marked accordingly, and analysis tools are applied over the measured features to see if the indications of disease progress improve. This system widespread, ease of use and availability is actually enabling the fast and efficient of new drugs introduction on the fields of Neurology and Ophthalmology.

### Slice Aggregation FF-SS-OCT:

For FF-SS-OCT system and method described above, to obtain high axial resolution, there is a need to have a wideband swept light source, however the wider the band of wavelength the higher the difficulty of implementation and cost of a laser based SS. Another issue relate to the high speckle noise associated with narrow band light source that is needed for providing instantaneous long coherence length. In the following embodiment we will show such wideband simplified FF-SS-OCT implementation.

In this embodiment we use non-coherent, narrowband SS illumination such that the instantaneous illumination spectral band is non-coherent but narrow band. Such light source would exhibit low coherence length, however much lower speckle associated noise, furthermore it may be better cost-effective and some engineering advantages. As a result of the short coherence length the working depth range is being narrowed, restricting depth working range. In order to extend the depth range, a slice aggregation method applied and the SS carried out in slices one after the other to obtain whatever needed wide depth range. There are some advantages to this method that will be explained below.

An adequate light source for this method can be obtained in a variety of realizations, The requirements are quite simple, a swept radiation source with narrowband temporal spectral profile that can swipe a broadband spectral range. For example, consider a 3 nm band source that can swipe it's center wavelength between 800 nm to 900 nm, the temporal coherence length determine the depth range under work, for example a source of ~3.2 nm band would exhibit coherence length ~100 μm in air, swiping this light source between 800 to 900 in steps of 2 nm will result axial depth resolution of ~3.2 um, using eq (1).

The light source may comprise a plurality of low coherence sources, such a source might be an array Light Emitting Diodes (LED) or a Superluminescent LEDs (SLED). It can also be any other technology for light radiating with low coherence that can be swept or change the central wavelength in a swipe. In FIG. 20 such a light source shown.

FIG. 20 illustrates a spectrum of a wideband Illumination source composed from an array of individual low coherence sources with moderate width. When activated—obtaining full-BW (2102) illumination source.

Practical example for this kind of light source is to create a Full BW source of 400 nm wide by using an array of 20 discrete sources (2101(1)-2101(N) assuming N=20) with each having a bandwidth Δλ=20 nm. The spacing between center wavelengths can be variable, also the bandwidth per source does not require to be equal. However, there may be some optimization that will benefit from a certain arrangement of the sources. Another consideration relates to the obtained full BW covered in a top-hat spectral profile. Or a gaussian shaped overall spectral profile. Typically, lasers are monochromatic, having much smaller bandwidth of 0.2-1 nm, however laser array or swept as light source may also be considered if it can obtain the desired spectral properties of the design.

Another optional light source realization can be a monochromator, comprising a broadband lamp or a LED, as input and generates monochromatic wavelength output, the output band is typically controlled via an exit slit and the temporal output center wavelength can be controlled externally by a controller.

Define N to be the number of discrete light source central wavelength. High-level sequence data capture sequence given by:
1. Set the reference arm to a position that its optical length equal to the z distance of the sample that is desired to scan.
2. Set the imaging optics focus distance also to the same z distance.
3. Activate first light source having λ₁ & Δλ₁),
4. Expose the image sensor array to the interference light during the source activation. The exposure time can be longer or shorter of the illumination pulse duration.
5. Capture the obtained sensor signal.
6. Repeat steps 3-4 above to capture 2 . . . N illumination wavelengths. It should be noted that the order of wavelength activation can be arbitrary.
7. Move the reference arm optical path length and the focusing distance to next z slice by increment of =ΔZ. The value of the depth step ΔZ discussed below.
8. Repeat the capture sequence above for this new position for 1 . . . M, where M is the number of desired depth slices.
9. Analysis:
   a. For each z slice swipe of frames—preform per pixel Inverse Fourier operation (over frames of different swept wavelength) to get the reflectivity profile per slice.
   b. Aggregate the z reflectivity slices for a complete 3D reflectivity where each slice offset by the appropriate ΔZ from its previous.

For simplicity of analysis, we assume all sources with the same bandwidth FWHM=Δλ, and the spacing also λᵢ−λᵢ₋₁=Δλ (i=2 . . . N). So, the obtained full bandwidth BW~=N*Δλ

For each light source, the coherence axial resolution given by:

$$Δ {z}_{i} = \frac{2 ln 2 {λ}_{i}^{2}}{π {Δλ}_{i}} ,$$

where i is the index of light source i=1 . . . N. (11)

Only within the common coherence length of all sources the depth discrimination is valid as it's desired to get high axial resolution. For good engineering practice one should choose

$$Δ {z}_{s} = {min}_{i} \{ Δ {z}_{i} \} ,$$

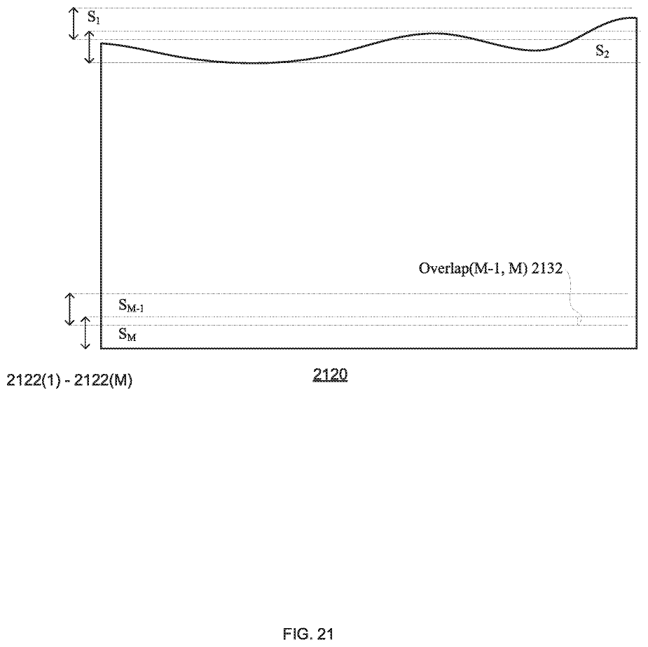

as the incremental slice step of reference OPL and focusing distance increments between swipes. Where it is guaranteed that all light sources are within the smallest range. Alternatively, for practical reasons one can choose a larger z step with some compromise of obtained axial resolution. FIG. 21 illustrates the slice aggregation depth range coverage, slice by slice (M slices denoted 2122(1)-2122(M)) with overlapping showing both coherence window and focus window aligned, slice by slice aggregation of depth data.

The overlap volume between slices S_{M-1} is and S_{M} denoted 2123). There is an overlap volume between each pair of consecutive slices. A slice is a 3D volume for which a 3D depth map is generated. The 2D depth map of a slice is obtained by executing one or more measurement sessions.

FIG. 21 illustrates a sample scanning description 2120, slice by slice with overlap, slice width should be smaller than the illumination coherence length and the DOF. Where the OPL and focus depth are aligned and move synchronically slice by slice. The number of slices is unbounded and can be adjusted for application needs as its limited by the sample optical penetration depth, or instrumentally by travel length of the reference OPL range and the optics focusing distance, in practice the tissue penetration depth will limit the slices.

This method exhibits several meaningful advantages relative to other OCT methods, one such advantage relates to the speckle noise associated with monochromatic illumination is the speckle contrast of spatial captured signal that is inversely related to the source spectral width (α 1/dλ). For practical SS the required depth range forces a narrowband swept laser to cover the desired depth range. But on the other hand, it exhibits high speckle contrast that deteriorate capture quality especially for in-vivo sampling where the mechanical jitters can't be avoided. The jitters cause for high sample to sample speckle noise—hence obscure the true signal. The method described here utilize relatively wider band sources (can be 3-20 nm) obtaining much lower speckle noise and lower coherence length that is being regained with multiple slices as described.

One more advantage relates to the computation needs, the required IFT decompositions typically need C=N*log(N) of mult+add where N is the number of wavelengths. Our embodiment utilizes stepwise small IFFT over small number of wavelengths the number of calculations would be C=M*N*log(N) Where M is the number of swipes.

For example consider a case of N=1000 in a SS-OCT=1000*log(1000)=3000 operations per axial mapping of a sample point. For our case assume N=10, and M=100 (M*N=1000, same number depth points) we get C=100*10*log(10)=1000, the computational complexity reduced by ⅓.

In vivo imaging of slice aggregation FF-SS-OCT method exhibit lateral and axial jitters between individual captured images. However, as discussed above each specific capture should have exposure time short enough to avoid image smearing. The images and depth slices can be registered by means of image processing methods to align back laterally the pixels and the axial reflectivity information.

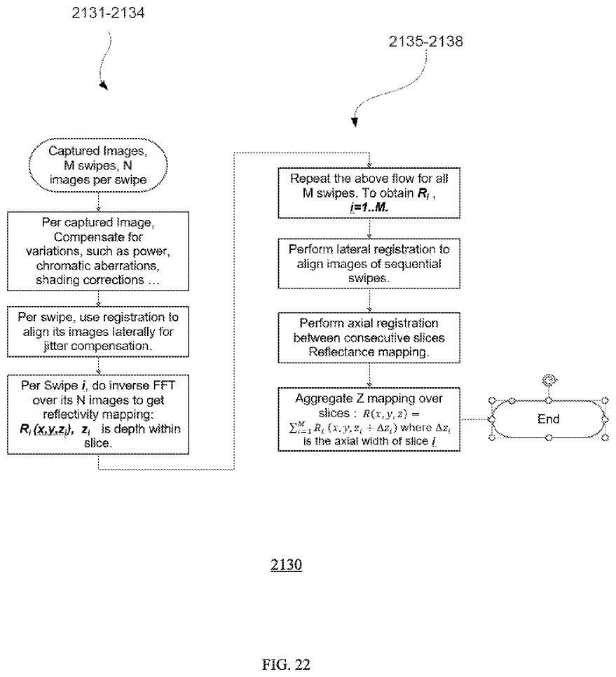

FIG. 22 describes the high-level processing flow 2130 for the proposed embodiment of a Slice Aggregation FF-SS-OCT method for in-vivo capture scenario. The processing flow performs lateral and axial alignment between images and reflectivity maps to compensate the jitters and other inaccuracies. Captured data from M depth slices (2131), each such slice contains N images of wavelengths swipe. Overall M*N images. The processing first compensates (2132) images for variations, for example in each image the light source emits light power that variate in time randomly due to noise, assuming there is a power measurement unit we can sample optical power per frame and compensate power mathematically to align all frames image power. The average power differences between the different wavelengths can be measured during calibration session pre-scanning. In addition, the images can be aligned regarding chromatic aberration, since images are with different wavelength there may be some small variation. Similarly other image signal processing (ISP) pipes can be used for image enhancements as known in the art of image processing and computer vision imaging system.

Further to image variations compensation, there is a step to align (2133) images for lateral jitter compensation using registration algorithm. Once images are aligned one can perform (2134) inverse Fourier transform, per pixel over the N wavelength. This results in the reflectivity mapping in z within the work z slice. The next N images are the consecutive swipe of wavelengths over the next depth slice. Repeating (2135) the Inverse Fourier Transform (IFT) for all slices would result with a set of reflectivity mapping over x,y field and the axial (z) axis of the slice {Rᵢ(x, y, zᵢ)}_{i=1 . . . M} next we need to align the swipes one to the other further compensating for jitters. The lateral slice alignment (2136) can be done over images of consecutive images, for example image of WL1 from slice 1 with same wavelength of slice 2 and so forth up to M. even though the focusing conditions changes from slice to slice it would be expected that registration still valid as images may be within DOF or very close. Another alternative is to do the slice to slice alignment using the reflectivity mapping, this way 3 axis alignment possible x,y,z. In order for the axial alignment (2137) to work well one would design the depth slices to have some overlapping, say ~20% overlap. The registration algorithm can receive the designed slice width thus it only needs to measure and compensate for the jitters and some slight other miss-accuracies. Following alignment, a aggregation over Z mapping (2138) along slices is provided.

In this context of discussion, the processing flow may include additional known measures for various compensations and enhancements, such as dispersion compensation, wavelength variations from ordered regulation compensation, denoising, rectification and so forth. These measures are not included in this description for brief and clear method explanation without diving into its fine details of implementation.

For a case of a transparent sample where the internal depth data is of interest, the basic SS-OCT method can't resolve the ambiguity of depths on the 2 sides of the 0 OPL difference as the inverse FT of a real signal exhibit symmetry around the center zero frequency. One way SS-OCT systems resolve this issue by using only ½ of the coherence sides. That is to set the zero OPL difference outside of the sample. This solution is undesirable for slice aggregation implementation as the method utilize multiple slices deep into the sample so there is no way to ignore one of the sides.

Another way to resolve this issue described at [13], where a phase modulation of the reference arm of π/2 (λ/4) cycle used.

Fourier Domain OCT is based on spectral interferometry, where recombined light from reference and sample arms is spectrally separated. The detected interference signal as function of k (k=2π/μ) may be expressed by [13]:

$${I}_{c o s} ( k ) = {I}_{r} ( k ) + 2 \sqrt{{I}_{s} ( k ) {I}_{r} ( k )} {\sum }_{n} {α}_{n} cos ( k {z}_{n} ) + {I}_{s} ( k ) \qquad ( 12 )$$

Where Iᵣ(k) and Iₛ(k) represent the intensities reflected from the reference arm and the sample arm respectively. αₙ is the square root of the sample reflectivity at depth zₙ where at z=0 is the sample z that is equal to reference arm OPL, the power spectra of IFT over I(k) would result ambiguous (zₙ, −zₙ) solution since the intensity is real number as the nature of FT. Adding a swipe over k with the addition of π/2 phase delay in the reference arm would result:

$${I}_{s i n} ( k ) = {I}_{r} ( k ) + 2 \sqrt{{I}_{s} ( k ) {I}_{r} ( k )} {\sum }_{n} {α}_{n} sin ( k {z}_{n} ) + {I}_{s} ( k ) \qquad ( 13 )$$

The combination of I(k)=I_{cos}(k)+iIₛᵢₙ(k), where i represent the imaginary part will produce a complex representation that will result a unique zₙ at the inverse FT transformation. However, this requires a second set of frames to be collected with the phase shifting.

Another issue is the DC component (at z=0) of the FT, as the Iᵣ(k) and Iₛ(k) parts can't be removed, traditional SS-OCT devices utilize balanced detection that removes the DC component of the signal. Allowing only the coherence signal for the capture, another approach is to subtract the reference spectral density profile form the interference density profile. In the following sections we will present a solution to these issues.

A different approach for DC component and complex conjugate (negative z) removal proposed at [14] by use off-axis angle between the sample and reference arm optical axis towards the sensor. This angle produces spatial fringes on the sensor surface that corresponds to the off-axis angle, the wavelength and the coherence amplitude. These spatial fringes are used for the DC and complex conjugate suppression. It was also shown that this method reduces autocorrelation noise (will be described below). The spatial fringe rate on the sensor surface is used by spatial or frequency filtering to extract the desired information. However, the method limits spatial work range considerably (by ~⅓) thus resolution affected and requires detection spatial response to be much higher than for on-axis method that is complex and costly.

### Autocorrelation Noise

Autocorrelation noise related to SS-OCT and SD-OCT refers to multiple reflections among reflecting elements in the sample, this noise described as autocorrelation noise. Depth information obtained by performing an inverse Fourier transform, yielding the following convolution.

$${\vert {FT}^{- 1} [ I ( k ) ] \vert}^{2} = {Γ}^{2} ( z ) ⊗ \{ δ ( 0 ) + {\sum }_{n} {α}_{n}^{2} δ ( z - {z}_{n} ) + {\sum }_{n} {α}_{n}^{2} δ ( z + {z}_{n} ) + O [ {I}_{s}^{2} / {I}_{r}^{2} ] \} \qquad ( 14 )$$

Where Γ²(z) represents the envelop of the coherence window the term δ(0) represents the DC component as the reference autocorrelation. The parts

$${\sum }_{n} {α}_{n}^{2} δ ( z - {z}_{n} ) and {\sum }_{n} {α}_{n}^{2} δ ( z + {z}_{n} )$$

are due to the interference between the sample arm and reference arm light, the part:

$$O [ {I}_{s}^{2} / {I}_{r}^{2} ]$$

describes the autocorrelation noise due to interference within the sample arm at the order of

$$[ {I}_{s}^{2} / {I}_{r}^{2} ] ,$$

one method to reduce the autocorrelation noise is by increasing the reference arm intensity Iᵣ relative to the sample arm, obtaining reduced ratio of

$${I}_{s}^{2} / {I}_{r}^{2} .$$

One can notice the autocorrelation noise is significant mostly for SS and SD OCT as the spectral split to narrow band samples exhibit a wide coherence length per band-slice, that is the coherence window Γ(z) is wide, thus exhibit autocorrelation noises in within its coherence range. That is not the case for this embodiment as the light source may feature smaller coherence length. A typical SS or SD-OCT can have a coherence length of 1-2 mm per band-sample, where for this embodiment the length may be 50-100 um, reducing considerably the autocorrelation noise as the coherence window is narrower.

Another important aspect of this embodiment is the use of relatively small slices of scan utilizing medium coherence length for each slice. This allows also to use high NA optics as the DOF can be as small as the coherence length, for engineering practice one would design the DOF to be somewhat longer than the coherence length. However other designs may be possible where the DOF is smaller or equal to the coherence length of the array of light sources. For continuous good quality z reflectivity mapping of a sample the spacing of z slices may be Sw=min(DOF, Coherence Length), adding some margins may be helpful for the aggregation of slices to full 3D sample reflectivity representation. Referring to the previous section discussing autocorrelation noise, a narrow DOF will further reduce the out-of-focus interferences. The work with low DOF requires the ability to modify the focusing window to follow the coherence window, this can be obtained via an alignment calibration procedure prior to the actual scan. But once having this capability in the device it enables to have high NA optics for obtaining high spatial resolution. Overall, this embodiment allows for simple, high NA, High resolution (diffraction limit), high axial resolution, shot noise limited, low autocorrelation noise, unbounded depth mapping of a sample.

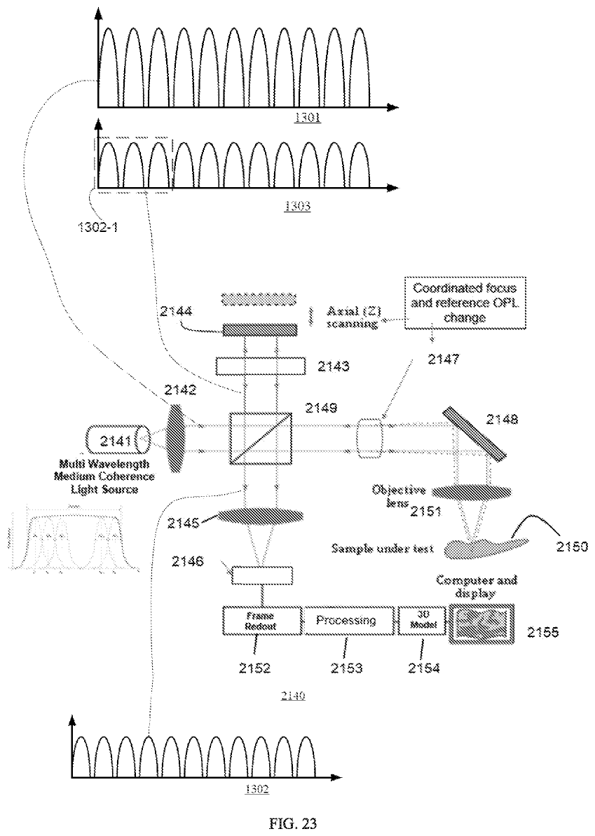

FIG. 23 illustrates the conceptual drawing of FF-SS-OCT system 2140 in accordance with this embodiment. Featuring the system elements described above. Field illumination with multiple wavelength light sources or a swiped source (2141) that are swept each depth slice, Full-Field imaging optics that comprises focus depth control optics (focusing optics 2147) and variable OPL reference arm (see the reference to axial scanning along z-axis) that comprises a phase modulator 2143. Interference light from both arms imaged onto 2D image sensor (2146) containing plurality of pixels that capture the sample. The capture of depth structure of the sample performed at slice-based sequence where both focusing window and coherence window aligned one to another. A phase modulator 2143 in the reference arm enable swipe repeat with π/2 phase relative to first swipe that results with the sin( ) component of the interferometry for the purpose of ambiguity resolving as discussed above. Focus depth control optics synchronized with reference arm OPL change. Step and wavelength swipe capture method. It should be noticed that the phase modulator component may be positioned in the sample arm as well without changing the outcome or quality. Also, different phase delays may be introduced with appropriate mathematical methods for ambiguity resolving. Regarding the processing flow, the phase delayed swipes images can be captured right after the non-delayed swipe or interleaved, that is for each light flash both phases can be captured before switch to the next wavelength, phase-delayed images can be aligned with the non-delayed images for in-vivo scanning to compensate the jitters in the lateral and axial direction prior to re-combining and depth reconstruction. The multiple wavelength light sources 2141 is followed by collimation lens 2142 that is followed by beam splitter 2149 that power splits the field illumination (radiation) between the measurement arm to the reference arm. FIG. 23 illustrates the power splitting—as sets of pulses (SOPs) 1301 is power split to first path SOPs 1303 and second path SPOs 1302. FIG. 23 also illustrates a SOP 1302-1 that includes three pulses of the same wavelength range. During the passage of the first pulse of SOP 1302-1 the phase modulator introduces a first delay (corresponds to a first phase), during the passage of the second pulse of SOP 1302-1 the phase modulator introduces a second delay (corresponds to a second phase), and during the passage of the third pulse of SOP 1302-1 the phase modulator introduces a third delay (corresponds to a third phase). The first phase, second phase and the third phase differ from each other.

It should be noted that pulses of a single SOP may be spaced apart from each other—and not adjacent to each other.

It should be noted that the phase modulator may be located at the measurement arm.

In the measurement arm the beam splitter 2149 is followed by focusing optics 2147, mirror 2148, and object lens 2151 (that focuses the radiation propagating in the measurement arm towards the sample under test 2150. Radiation from the sample under test propagates to the objective lens, the mirror, the focusing optics, and to the beam splitter 2149 to form an interference pattern.

In the reference arm, the beam splitter 2149 is followed by phase modulator 2143 and by the reference mirror 2144 that is movable along the Z axis to provide different delay values (OPL values). Radiation from the reference mirror 2144 propagates to the phase modulator and reaches the beam splitter 2149 to form the interference pattern.

The interference pattern is imaged by the imaging optics 2145 onto the 2D sensor array 2146. The 2D sensor array 2146 is read by frame readout 2152, a frame (an image) is processed (2153) in accordance to the OCT processing flow discussed above and information obtained from multiple frames (from one or more measurement sessions) are processed to provide depth information such as a 3D map, which may be stored, transmitted or displayed (on display 2155).

Slice Aggregation SS FF-OCT with 3 or More Phase Delays (Hybrid FF-OCT):

Further improvement to the slice aggregation FF-SS-OCT can be obtained by adding an additional phase or more, as the I_{cos}(k) and Iₛᵢₙ(k) contain a CD component that need to be removed or accurate depth calculations. The DC removal in the 2-phase case described above might introduce noise to the calculations due to temporal or wavelength based variations.

A 3 phases can be utilized with phases of 0, π/2 and π. The π phase is inversed to the cos( ) term define the subtraction of 0 and π phases by knowing that cos(kz+π)=−cos(kz), define:

$${I}_{D C} ( k ) = {I}_{c o s} ( k ) - {I}_{- c o s} ( k ) = 4 \sqrt{{I}_{s} ( k ) {I}_{r} ( k )} {\sum }_{n} {α}_{n} ( cos ( k {z}_{n} ) ) \qquad ( 15 )$$

As I_{DC}(k) is the differential cos( ) coherence amplitude. The DC component is given by:

$$D C ( k ) = {I}_{r} ( k ) + {I}_{s} ( k ) = [ {I}_{c o s} ( k ) + {I}_{- c o s} ( k ) ] / 2 \qquad ( 16 )$$

And the sin( ) differential coherence amplitude

$${I}_{D S} ( k ) = 2 * ( {I}_{s i n} ( k ) - D C ( k ) ) = 4 \sqrt{{I}_{s} ( k ) {I}_{r} ( k )} {\sum }_{n} {α}_{n} ( sin ( k {z}_{n} ) ) \qquad ( 17 )$$

Resulting I_{DC}(k) and I_{Ds}(k) gives the cos( ) and sin( ) coherence amplitude needed for depth reconstruction. Notice the I_{DS}(k) value is factorized by 2 relatives to I_{DC}(k) to balance its power with I_{DC}(k), There is no loss of efficiency or power waste due to the addition of the third swipe as they are part of the calculation. The depth reconstruction is than carried out by:

$$\begin{matrix}R ( z ) = {\vert {FT}^{- 1} [ {I}_{D c} ( k ) + {iI}_{D s} ( k ) ] \vert}^{2} \\ = {\vert {FT}^{- 1} [ 4 \sqrt{{I}_{s} ( k ) {I}_{r} ( k )} {\sum }_{n} {α}_{n} exp \{ ik {z}_{n} \} ] \vert}^{2}\end{matrix} \qquad ( 18 )$$

In the above P=3 phase case we used 0, π/2 and π, it can be shown that there are other phase selections can be chosen such as 0, 2π/3 and 4π/3 that are evenly spaced, any arbitrary 3 or more phase distribution that map the space can be chosen.

Considering a case of 4 node sensor, 4 phases can be used such as: 0, π/2, π and 3π/2. The 4 phases map to I₀(k)=I_{cos}(k), I_{π}(k)=I_{-cos}(k), I_{π/2}(k)=Iₛᵢₙ(k), I_{3π/2}(k)=I₋ₛᵢₙ(k) Resulting the differential coherence terms:

$${I}_{D c} ( k ) = {I}_{0} ( k ) - {I}_{π} ( k ) = 4 \sqrt{{I}_{s} ( k ) {I}_{r} ( k )} {\sum }_{n} {α}_{n} cos ( k {z}_{n} ) \qquad ( 19 )$$

$${I}_{D s} ( k ) = {I}_{π / 2} ( k ) - {I}_{3 π / 2} ( k ) = 4 \sqrt{{I}_{s} ( k ) {I}_{r} ( k )} {\sum }_{n} {α}_{n} sin ( k {z}_{n} ) \qquad ( 20 )$$

And using eq (18) for depth resolving.

The 4 phases split to their cos( ) and sin( ) terms, differentiated (I_{Dc}(k), I_{Ds}(k)) are measuring the modulation amplitude and phase over k, also expressed in the imaginary exponential term exp{ik zₙ}. So, this method is a hybrid of Time-Domain and Swept-Source, benefiting from both methods. Following the above analysis, it can be further generalized to any number of phases P=3 and above. P>=4 is overdetermined, helping to improve SNR, where P=3 necessary for explicit sin( ) and cos( ) coherence amplitude obtain by enabling direct removal of the DC(k) component. The phase modulator switches in time between the P phases, in discrete steps as much as possible, where the sensor exposure timing would need to be accurately aligned with is. However, due to electronic delays of the components there may be miss-alignment between sensor and modulator in time. This instrument response timing delay can be easily measured and compensated via a calibration process. Each swipe in the k domain of the various wavelengths is done in stationary motionless conditions of the reference OPL, meaning only the stabilization time and instrumental timing delays involved. The exposure timing for each capture of a frame can be designed to start after stabilization of the phase. In this method each sensor exposure collects light of single phase and wavelength.

### In-Vivo FF-OCT Axial Jitters Reduction and Compensation.

The basic feature that differentiates In-Vivo sample from other sample types is that the imaging device, such as the OCT device can't be rigidly connected to the sample, thus the device should be able to be robust for jitters, in the OCT case it affects not only the placement and focusing quality, but it may also affect depth reconstruction accuracy and the axial and lateral contrast. As the vibrations change the OPL from the sample but does not change the OPL at the interferometer reference arm. This issue may also appear when using hand-held device that is not in rigid contact with the sample where the sample can be any material or specimen under test that may not be confined to in-vivo sample.

In this section additional improvements for OCT imaging and depth information quality presented for in-vivo OCT capture, as discussed above, each image capture of the FF-OCT can be at a short duration so there are no substantial jitters during each frame capture that might cause for contrast degradation, however the complete OCT mapping requires multiple frames capture, all FF-OCT methods described above require plurality of frames. Lateral jitters can be fixed by means of registration to stabilize images laterally, numerous image stabilization processing pipes are available by application processors for that purpose. Regarding axial jitters (along the optical axis) there is a need for motion compensation to get accurate depth measurements. For example, the retina internal layer thickness is an important measure for many diseases and the main use of retinal OCT. In this embodiment we may incorporate a distance measurement sensor that measures the distance between the device and the in-vivo sample under test. These distance measurements between the device and sample are captured simultaneously to OCT frames and are being used for depth data compensation. For most use cases these axial jitters have low frequency at the range of 5-20 Hz at its spectral energy distribution, where the OCT frame rate may be 100-500 fps. So, the distance variation may be easily compensated once sampled fast enough and measured accurately. In the case of FF-TD-OCT the depth of each capture may be corrected by the distance measure such that the OPL is corrected for obtaining accurate depth value per frame.

For FF-SS-OCT the distance measurement can be used to phase correct the frames, as discussed above, each frame capture ideally should be in a constant OPL as the reference arm is stationary, but due to the jitters the sample arm OPL vibrates, thus introducing noise and contrast reduction. These OPL variations due to axial jitters can be characterized as a phase delay to the interference correlation function that is e^{-ik(d(t)-d}^{₀}⁾ where d(t) is the temporal axial jitter variations as given from the optical distance measurement device, and d₀ is the distance measure of the first captured frame. Hence the first captured frame in the SS sequence can serve as reference such that the next sequential frames are compensated to it. Compensation performed by the complex conjugate of each frame by multiplying it by: e^{+ik(d}^{ₙ}^{-d}^{₀}⁾, where n represents the frame number and dₙ is the distance measured between the device and the sample during the n^{th} frame.

The Hybrid FF-OCT can utilize both above measures for axial jitters compensation, each depth slice, is a swept radiation source sequence that can be fixed as described above using the complex conjugate. Where the variations between slices can be compensated for the OPL variation as described for the TD method.

The optical distance measurement device can utilize various methodologies such as Time-of-Flight or triangulation based such as stereo pair, assisted stereo or structured light or any other distance measurement technology that meet the following requirements:
1. Distance measurement accuracy and repeatability adequate for maintaining overall output depth accuracy and axial contrast of the device.
2. Fast capture in synchronization the OCT frames such that each frame can be paired with its time match distance measure.
3. No interference between both distance and OCT captures, may be temporal, spatial or wavelength-based differentiation.
4. Firm mechanical mount of the optical distance measurement device to the OCT device for the measurement would represent true OPL jitter induced variations as much as possible.

The actual device, measurement, activation and mounting methods are application dependent. For example, the eye retina OCT one can use an optical distance measurement device that observe the patient forehead distance variation from the device. As we are only interested in the OPL variation during the OCT scan. With the assumption that the patient whole head is approximated to a rigid body than the forehead axial distance vibrations are representative for the eye to device OPL variations we look for.

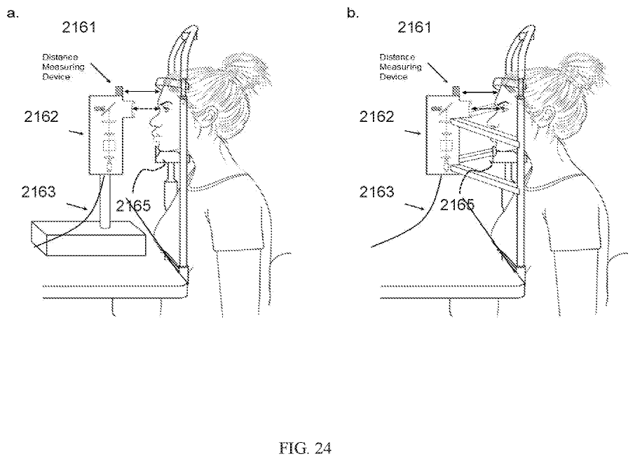

FIG. 24 part (a) illustrates a possible embodiment of ophthalmology OCT device 2162 incorporating a optical distance measurement device 2161 on its top casing. The distance measuring device is rigidly mounted onto the OCT device and provides distance readings to the patient forehead during frames OCT frames capture. Where its measurements are used for depth accuracy improvement and contrast enhancement. FIG. 24 part (b) further illustrates rigid attachment to the patient chinrest 2165 as a measure to reduce jitters. FIGS. 24 part (a) and 24 part (b) also illustrates a communication lines 2163 that facilitate communication with the ophthalmology OCT device 2162.

Most probable the measuring device is optically based; cross noise interference might occur. Thus, some separation is needed. In this example, the distance measuring is spatially separated from the imaging optics, so there is no risk of interference. For the more general case, in other embodiments or use cases, the distance measuring method may use the same optical path of the imaging device where the separation can be in time, the distance is being measured at a time gap between imaging exposures, or by wavelength separation and a filter to block undesired wavelength at each modality optical path.

### Jitter Compensation Using OPL Compensation Component.

Another measure for axial jitters reduction can be obtained with an OPL controlled component that may be connected to the distance measuring device. It may be a pocket cell, or a liquid-crystal, sliding wedge or any other known component that can compensate for a major part of the jitter as being measured by the optical distance measurement device. The OPL stabilization component may be located at the reference arm or sample arm of the interferometer. The advantage is that the jitter compensation obtained via optical measure improves the sampled signal quality. Contributing to the contrast enhancement quality and depth estimation accuracy, also reducing the needed processing complexity.

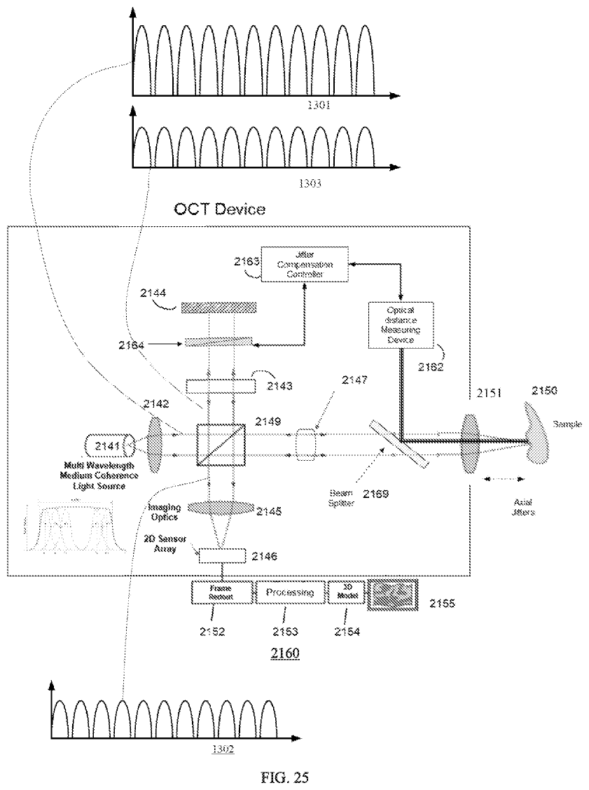

FIG. 25 illustrates a schematic OCT device example comprising OPL compensation system that includes an optical based distance measurement that pass through a beam splitter onto the inspected sample via sample arm optics, back and forth, a sliding wedge OPL compensator module and a real-time controller that reads the jitter based distance variation and activate the slider wedge accordingly

FIG. 25 illustrates a possible example of an OCT device comprising OPL compensation system that includes an optical based distance measurement module that pass through a beam splitter onto the inspected sample via sample arm optics, back and forth, a sliding wedge OPL compensator and a real-time controller that reads the jitter-based distance variation and activate the slider wedge accordingly. The separation between the OCT imaging process and the distance measuring can be time based or wavelength based, where at wavelength separation case the beam splitter may be a dichroic splitter that is reflective to the OCT wavelength and transparent to the distance measuring illumination wavelength. There are many commercial accurate and fast response such sensors. The OPL compensation by a slider wedge is a possible embodiment, where it can be any optical device that may do the needed functionality, response and cost/size considerations.

The SS light source 2141 is followed by collimation lens 2142 that is followed by beam splitter 2149 that power splits the field illumination (radiation) between the measurement arm to the reference arm. FIG. 25 illustrates the power splitting—as sets of pulses (SOPs) 1301 is power split to first path SOPs 1303 and second path SPOs 1302.

In the measurement arm the beam splitter 2149 is followed by focusing optics 2147, beam splitter 2169, and object lens 2151 (that focuses the radiation propagating in the measurement arm towards the sample under test 2150. Radiation from the sample under test propagates back to the objective lens, the mirror the focusing optics, and to the beam splitter 2149 to form an interference pattern. In the reference arm, the beam splitter 2149 is followed by phase modulator 2143, a compensation module such as sliding wedge OPL 2164, and by the reference mirror 2144 that is movable along the Z axis to provide different reference delay OPL values. The sliding wedge OPL 2164 is also configured to introduce different delay values. Radiation from the reference mirror 2144 propagates to sliding wedge OPL 2164, the phase modulator and reaches the beam splitter 2149 to form the interference pattern.

The interference pattern is imaged by the imaging optics 2145 onto the 2D sensor array 2146. The 2D sensor array 2146 is read by frame readout 2152, a frame (an image) is processed (2153) and information obtained from multiple frames (from one or more measurement sessions) are processed to provide depth information such as a 3D map 2154, which may be stored, transmitted or displayed (on display 2155).

In addition to the OCT device there is also a distance (axial distance) measurement and compensation system that includes optical distance measuring device 2162 (that includes a dedicated light source), and a jitter compensation controller 2163 that is configured to control the compensation module such as sliding wedge OPL 2164 to compensate for axial distance changes—as measured by the optical distance measuring device 2162.

In FIG. 25 the OCT device and the measurement and compensation system share a beam splitter. Additionally, the OCT device and the measurement and compensation system may not share any optical components. Additionally, the OCT device and the measurement and compensation system may share any other component and/or may share multiple components.

### Jitter Compensation and Contrast Enhancement Using OCT Self-Optics.

SS-OCT (and FD-OCT) axial signal quality, meaning contrast, resolution, and SNR, affected by motion jitters during wavelength swipe of the source at the level of a fraction of the wavelength. Typically, it is assumed the OPL is constant during the swipe since it is carried out using a stationary system and sample. There are some algorithmic methods for jitter restoration and constant enhancement. One approach described above for the FF-SS-OCT, but it might not be suit for the Hybrid FF-OCT presented in this embodiment. In this section we will show a new way to estimate the jitters accurately in a sub-wavelength resolution.

The SS-OCT signal with temporal dependance given by:

$${I}_{c o s} ( k , t ) = {I}_{r} ( k ) + 2 \sqrt{{I}_{s} ( k ) {I}_{r} ( k )} {\sum }_{n} {α}_{n} cos ( k ( {z}_{n} + d z ( t ) ) ) + {I}_{s} ( k ) \qquad ( 21 )$$

Using 4 phases shift captures of 0, π/2, π and 3π/2. The amplitude and phase of the coherence signal over time is expressed by adding the time components to (17) and (18).

$${I}_{D c} ( k , t ) = {I}_{0} ( k , t ) - {I}_{π} ( k , t ) = 4 \sqrt{{I}_{s} ( k ) {I}_{r} ( k )} {\sum }_{n} {α}_{n} cos ( k ( {z}_{n} + d z ( t ) ) ) \qquad ( 22 )$$

$${I}_{D s} ( k , t ) = {I}_{π / 2} ( k , t ) - {I}_{3 π / 2} ( k , t ) = 4 \sqrt{{I}_{s} ( k ) {I}_{r} ( k )} {\sum }_{n} {α}_{n} sin ( k ( {z}_{n} + d z ( t ) ) ) \qquad ( 23 )$$

The different phases capture sequence should be fast enough relative to the motion jitters such that in this context we consider the temporal axial jitter dz(t) as the time of the capture of all phases of the same wavelength participating in the measurement, in this case its 4 phases frames. This can be accomplished also by a multi-node sensor where each node accumulates charges of different phase carried out in fast capture session. And, in the extreme case fast phase modulation during the pulse with multiple accumulations of equal phase timing, capturing the different phases to different nodes interleaved at the same time such there will not be any axial jitters among the different phases captured frames.

The jitter value, expressed by dz(t) can be estimated at time interval between t₁ to t₂ and Δz=z(t₂)−z(t₂), if we assume the time elapsed is short enough for the condition |kΔz|<π fulfilled we can have an accurate estimate for the jitter by capturing 2 consecutive frames, first frame captured at t₁ and the second at t₂ where each frame comprises 4 phases subframes. Both frames at the same wavenumber k. Decomposition of eq. (22) and (23) to its trigonometric identities would result the following:

$$[ \begin{matrix}{I}_{D s} ( k , {t}_{2} ) \\ {I}_{Dc} ( k , {t}_{2} )\end{matrix} ] = [ \begin{matrix}cos φ & sin φ \\ - sin φ & cos φ\end{matrix} ] * [ \begin{matrix}{I}_{D s} ( k , {t}_{1} ) \\ {I}_{Dc} ( k , {t}_{1} )\end{matrix} ] \qquad ( 24 )$$

That is the coherence signal obtained due to axial jitter exhibit a phase delay given by the phase φ=kΔz, this phase delay matrix is known for also as geometric rotation matrix of a 2d object, numerous methods can be used for rotation angle φp estimation.

Practical usage of this method for jitter noise compensation would be to incorporate repeated capture of a same wavelength in each swipe of the swiped source of a slice capture, the repeated capture rate of the same wavelength depends on the expected jitter frequency and amplitude of the device vs sample, for accurate phase correction one would design the repeated capture at a rate that guarantees estimation without danger of ambiguity and large interpolation errors. Furthermore, the captured signals I_{Dc}(k, t) and I_{Ds}(k, t) used for the phase estimation can be average over a large plurality of pixels to remove temporal and other noise sources.

$${I}_{Dc} ( k , t ) = \frac{{\sum }_{x} {\sum }_{y} {I}_{Dc} ( k , t , x , y )}{{N}_{x} {N}_{y}} \qquad ( 25 )$$

$$and$$

$${I}_{Ds} ( k , t ) = \frac{{\sum }_{x} {\sum }_{y} {I}_{Ds} ( k , t , x , y )}{{N}_{x} {N}_{y}}$$

Where Nₓ and N_{y} are the number of participating pixels in x and y axes of the sensor, the region can be a subset of pixels in the center of the image, or the whole sensor pixels. Furthermore, in case the jitters also having a tilt component, meaning that the sample axal variations differ over frame, this analysis may be carried out at several regions over the sensor each one giving its z displacement that can be used over the entire frame by interpolation or extrapolation.

Once axial displacement estimation obtained, the mathematical compensation the displacement can later be done by phase correction of each wavelength sample by e^{-ik(z(t))} prior to IFT operation, were z(t) obtained by interpolation between the consecutive samples carried out using the same wavelength that are used for the jitter estimation. Simple implementation would be a linear interpolation for each wavelength capture depending on its timing relative to the same wavelength sample timing.

One of the strengths of this proposed method is that the coherence amplitude captures by using 4 phases can be done simultaneously using a multi node sensor that may capture the phases concurrently using fast phase modulation. The concurrent phases capture guarantees the jitter between the 4 captures is practically zero. Same for the differential signals I_{Dc} and I_{Ds} that eliminate the DC component completely due to the concurrent capture. Also, power variation noise minimised.

It should be noted that this method description we assumed capture of 4 phases per wavelength, but it can be applied for 3 or more optical phases where it can be shown the axial caused phase change of the coherence signal can be reconstructed. Case of 2 optical phases the solution ambiguity reduces to the range of |kΔz|<π/2 (instead of |kΔz|<π) with shortfalls of less noise immune to DC variations and the DC component would need to be estimated using additional methods. However, once the DC component estimation obtained, we can apply this method for jitter compensation as well.

### Hand-Held Portable FF-OCT Device

FF-OCT proposed in this embodiment comprises a device and methods to implement OCT while using 2D imaging of the entire sample surface. This enables to overcome high jitter and large hand motions typically associated with in-vivo imaging. During the capture sequence the captured data may exhibit large frame to frame variations, both axial and lateral. The OCT device may be mounted on the patient sampled area such as skin or retina, the sequence OCT images may miss-aligned due to instability of the tester's holding arm during the sequence. The sampled surface area should be large enough such that most images would have common coverage area, and OCT map of the common area can be generated. In regard for axial jitters, the compensations described above may be utilized for the purpose of obtaining good quality of OCT data to be delivered.

Optical alignment system (described below) may be added to control the beam optical path to compensate cases where the beam of light needs to pass through narrow apertures such as the eye iris at the case of ophthalmology measurement.

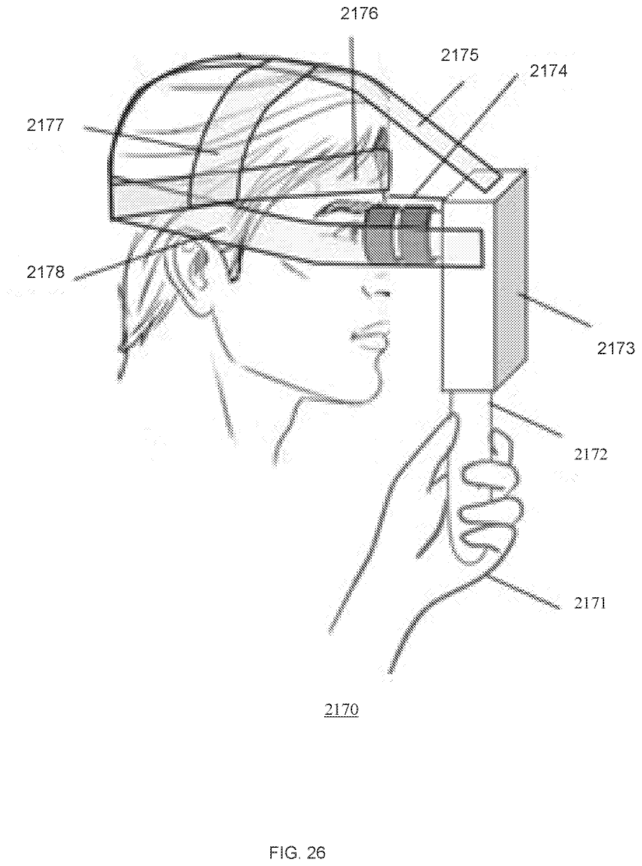

Additional helper for the hand-held device may be a mechanical holder that would help reducing jitter. The OCT device may connect to a helmet that would hold the device steady in front of the patient eye in case of ophthalmology test or ear test. And strings to the patient body in case of other body organ. This mechanical attachment would not completely remove the motions and jitters however may reduce it to acceptable level that the compensations would work adequately. FIG. 26 illustrates a person that holds (by hand 2171) the handle 2172 of a OCT device that includes a body 2173 and optics/eye interface 2174. The body 2173 is held by stripes (such as lower longitudinal stripe 2178, hear surrounding band 2176, upper stripe 2175) that partially surround an upper part of a head of the person. Alternatively the device 2173 handle 2172 can be held by the testing person.

### Hybrid FF-SS-OCT Using Phase Modulation and Multi-Node 2D Sensor.

Further improvement to the slice aggregation FF-SS-OCT can be obtained by using a multi-node sensor described above for FF-TD-OCT. The hardware configuration for the Hybrid OCT method described at FIG. 4 also applicable here where the sensor is a multi-node type, the operational method is somewhat different. The usage of multi-node sensor can further improve in-vivo OCT signal quality by reducing the time between concurrent capture of the P phases.

The Hybrid OCT method described above involves capturing a series of swipe wavelength frames where for each wavelength, multi-phase frames are needed. However, for In-Vivo OCT axial jitters is a dominant noise source. Multi-node sensor enables concurrent capture of the phases one after the other at the same light pulse, thus reducing system oriented noise sources.

In this embodiment we may use fast phase modulation and multi-node sensor at 2 possible methods:
Per wavelength k and P phases, a sensor with P nodes may capture the P phases one after the other with minimal time gap between phases. For example, for 1 ms exposure each phase activated for duration of is active 250 us, ignoring phase and node switching time. Actual case can, assuming 50 us can be 200 us exposure to each phase and 50 us switching gap. The Multi-Node senso allow for fast capture of P images with minimal time gap in between.
Fast cyclic modulation at high frequency and of the optical phase and P (P>=3) node aggregation in each modulation cycle. This homodyne demodulation, obtaining concurrent fast phase captures, without any time gap between frames due to readout. The method described in detail for the FF-TD-OCT above. This method captures the phases concurrently so that system and mechanical jitter induced noises are zero. For example The phase modulation can modulate the reference arm path for example at 100 kHz and the sensor nodes can aggregate phases in homodyne manner integrate multiple phases into phase nodes, each node (bucket) aggregates one phase. For a 4-nodes (P=4) pixel each phase may collect ¼ cycle, that is 2.5 us duration and aggregate over multiple cycles. In this case the coherence amplitude and phase can be decomposed to their cos( ) and sin( ) components and later used for the depth reconstruction using eq. (18).

### OCT Angiography

Optical Coherence Tomography Angiography (OCTA) is a non-invasive imaging technique used primarily in ophthalmology. It's an advanced form of Optical Coherence Tomography (OCT) that provides high-resolution three-dimensional images of the retina and choroid, which are layers at the back of the eye. OCTA works by using light waves to take cross-sectional pictures of the retina. It allows for the visualization of blood flow in the retinal and choroidal vessels without the need for dye injections, which are commonly used in other types of angiography. This makes OCTA safer and more comfortable for patients.

The technology is particularly useful for diagnosing and monitoring diseases that affect the blood vessels in the eye, such as diabetic retinopathy, age-related macular degeneration, and glaucoma. It helps in detecting abnormalities in the vascular network and can provide insights into disease progression and treatment efficacy.

OCTA blood flow detection relay upon the speckle variation of light reflection from the samples surface such as the retina due to blood cells motion in the blood vessels. The method comprises repeated OCT scans for a duration long enough to detect the amplitude variations of the OCT information and to enhance signal to noise.

In this embodiment fast OCTA may be implemented by using FF-OCT methods presented (FF-SS-OCT or FF-Hybrid-OCT). The concurrent temporal image capture of sample surface captures enables the usage of very effective methods for signal enhancements and noise suppression that can provide superior resulted blood flow and vessel mapping in much faster time of capture and analysis. Using the following flow:
1. Perform repeated OCT cycles N times in accordance to one of the OCT methods described in this embodiment.
2. Generate 3D reflectivity map per cycle.
3. Align the N maps to one another using a method such as correlation to register all N maps to a common 3D coordinate system.
4. Find areas within the 3D map with high reflectivity fluctuations over time (the N repeated OCT maps). Using one or more of the following methods:
   a. Enhance areas with high temporal fluctuations. Such as high variance over time.
   b. Enhance temporal harmonic variation that correlates to the actual heart rate or its harmonics. Such as a correlation calculation or Fourier spectral transformation.
      i. Spectral Amplitude of rate that correspond to the subject heart rate.
      ii. Spectral Amplitude of rate that correspond to the adjacent volume elements.
   c. Enhance neighboring volume elements that fluctuate at the same rate and phase as blood vessels are typically tubes that pass the blood cells inside.
   d. Suppression of noise data that fluctuate randomly in time or space. The suppression may use adaptive filtering such as Kalman filter or any other noise suppression filtration.

It should be noted that some of these signal enhancements and noise suppression methods are only possible since the data capture occurs concurrently such as the OCT methods proposed in this embodiment.

Using of fast OCT captures of the N repetitions is required for an optimized OCTA performance so that the blood flow fluctuations rate are sampled adequately. Each OCT duration time should be smaller than ½ of the heart rate cycle as required by Nyquist sampling theorem. In order to facilitate this requirement, the light source swipe range can be accommodated to a matching capture sequence. Another option to obtain fast OCT capture may be by reducing the spatial resolution because many of the sensors are data bandwidth limited. This may result lower axial resolution or lower axial resolution or both for the OCTA mapping relative to regular single OCT capture in a realistic device.

### Blood Flow and Phase and Amplitude Propagation

As described above, the concurrent full field OCT capture enables also an analysis of the blood flow propagation within the sampled surface. Spectral analysis and/or correlation method can provide rate and phase information per segment region. This information can be used for generating phase or amplitude propagation maps, similarly to topographic maps where equal phase lines are sowing the propagation of blood flow within the sample. The method steps are:
1. Follow OCTA capture sequence and analysis for generating 3D blood vessel enhanced mapping.
2. Split the volumetric data to localized segments, each segment relates to a region in the 3D information. The segment assignment method may be arbitrary selected. For example:
   a. Split to 3D volume element cubes array with arbitrary dimensions.
   b. Split to specific blood vessels for certain length.
3. Per segment—find the amplitude and phase of signal variations that best characterizes blood flow blood flow within the segment.
4. Generate phase and amplitude mapping of the entire 3D information. This can be done by connecting neighboring segments with equal or similar phase or amplitude.
5. Optionally: Apply enhancements or smoothing methods for reducing noise and improve information.
6. Generate phase propagation 3D map that will contain the information of how blood propagates in the sample 3D space.

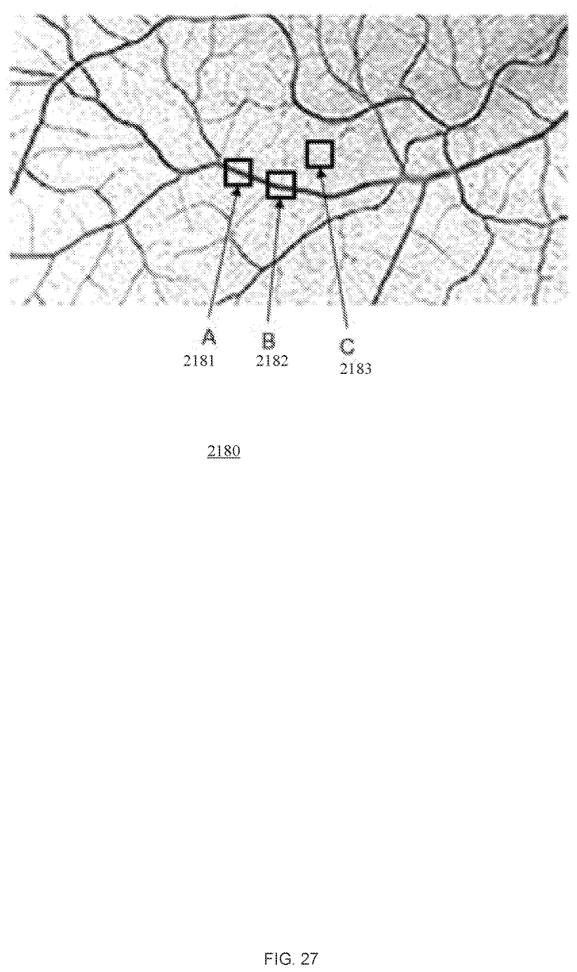

FIG. 27-29 illustrate the identification of blood vessel, the amplitude and the phase of flow. FIG. 27 is a 2-dimensional representation of the 3-dimensional blood vessel map of an in-vivo biological tissue, segments A 2181, B 2182 and C 2183 marked, where segments A and B marking a blood vessel in neighboring positions, segment C is nearby A and B but isn't a blood vessel segment.

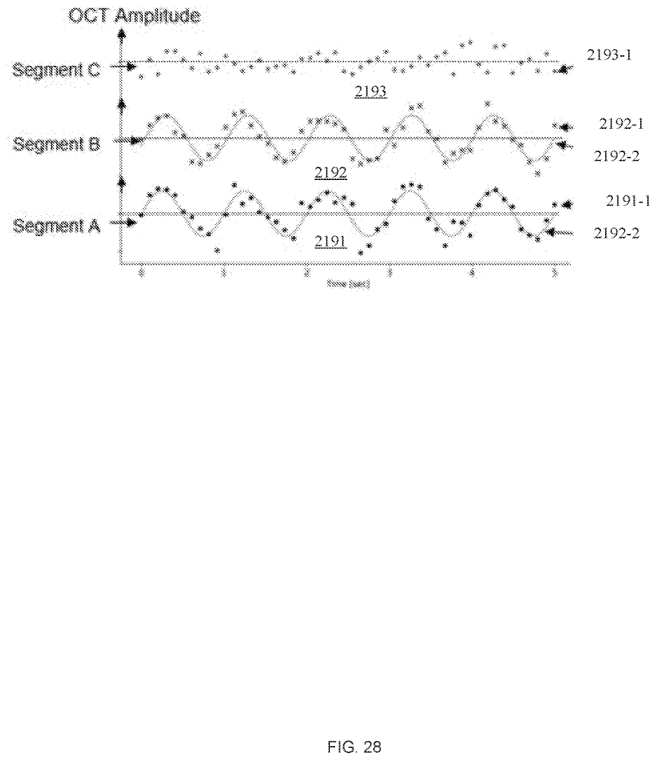

FIG. 28 showing the corresponding OCT amplitude over time of segments A (2191-1), B (2192-1) and C (2193-1). The OCT reading of segment A and B exhibit cyclic pattern that corresponds to the cyclic rate that may be the heart rate. The main harmonic cycle of A (2191-2), B (2192-2) illustrates the main harmonic rate of the readings 2191-1 and 2192-1, the actual OCT amplitude estimation include added measurement noise that is the major deviation from the pure cyclic shape. Segment C readings (2193-1), does not correlate to any harmonics, or the harmonic amplitude power is very low relative to the measurement noise, so it reflects noise mainly. The rate, amplitude and phase of each segment can be detected using a spectral analysis such as Fourier transformation or correlation with expected cyclic rate the analysis can also enhance neighboring segments that beat at the same rate and similar phase. This cyclic behavior and neighboring correlation can provide significant means for noise reduction and enhancement. Segments that do not contain blood vessels are expected to have poor harmonic correlation that is low cyclic behavior and could be easily filtered as noise to obtain high quality blood vessel OCTA 3-D mapping.

Segment A and B location on the same blood vessel, it's expected that the cycle of both is identical but the phase will represent the blood flow velocity and flow direction in the vessel. If the A segment phase precedes B, we can deduce that flow is from A to B and vice versa. More ever the topographic representation of blood flow can be made.

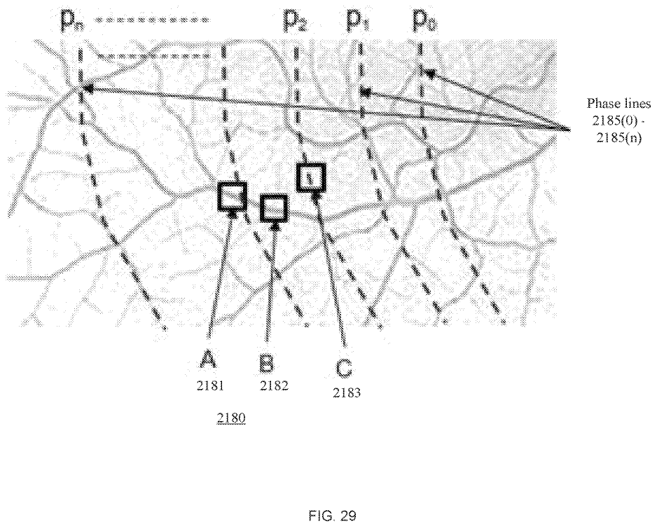

FIG. 29 illustrates 2-Dimensional topographic representation of blood flow overlayed over the 2-D of the blood vessels map. Lines P₀, P₀ . . . Pₙ (phase lines 2185(0_2185(n)) represent equal phase lines that may show blood flow direction and velocity. It should be understood that the 2-D representation is for illustration purpose where the actual mapping and analysis is 3-D.

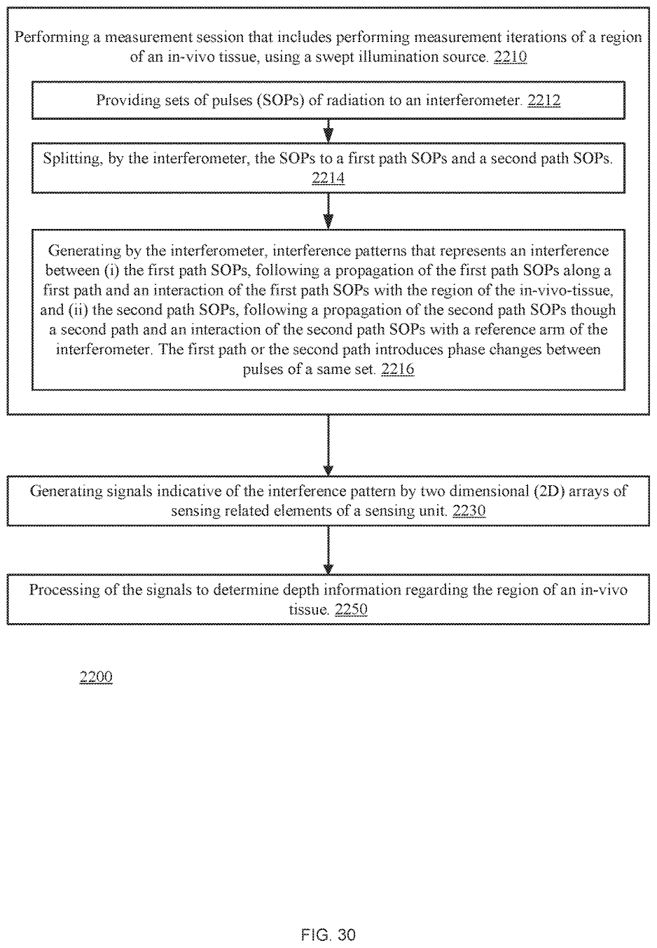

FIG. 30 illustrates an example of method 2200 for optical coherence tomography (OCT). According to an embodiment, method 2200 includes:

Step 2210 of performing a measurement session that includes performing measurement iterations of a region of an in-vivo tissue, using a swept illumination source. A swept illumination source may be implemented in various manners—using an adjustable wavelength illumination source or using different illumination elements and selecting between them. A swept illumination source is configured to transmit at a point in time radiation of a narrower wavelength width than the width of an overall wavelength range that eventually illuminates the region. S, for example FIG. 20.

Step 2230 of generating signals indicative of the interference pattern by two dimensional (2D) array of sensing related elements of a sensing unit.

Step 2250 of processing the signals.

According to an embodiment, step 2250 includes processing of the signals to determine depth information regarding the region of an in-vivo tissue.

According to an embodiment, each measurement iteration (of step 2210) includes:
(a) Providing (2212) sets of pulses (SOPs) of radiation to an interferometer. Pluses of each SOP have a same wavelength range, and at least some of the SOPs differ from each other by their wavelength range.
(b) Splitting (2214), by the interferometer, the SOPs to a first path SOPs and a second path SOPs. According to an embodiment the splitting is a power splitting—each SOP is power split to two weaker pulses. For example—the number of SOPs provided to the interferometer equals the number of the first path SOPs and equals to the number of second path SOPs.
(c) Generating (2216) by the interferometer, interference patterns that represents an interference between (i) the first path SOPs, following a propagation of the first path SOPs along a first path and an interaction of the first path SOPs with the region of the in-vivo-tissue, and (ii) the second path SOPs, following a propagation of the second path SOPs though a second path and an interaction of the second path SOPs with a reference arm of the interferometer. The first path or the second path introduces phase changes between pulses of a same set.

According to an embodiment, the at least one set of pulses includes three pulses.

According to an embodiment, the interferometer belongs to a hand held OCT device. See, for example, FIG. 26.

According to an embodiment, the method includes sensing different pulses of a set of pulses by different accumulation elements of a 2D array of the sensing unit. See, for example the sensor of FIG. 7.

According to an embodiment, the method includes introduces a lateral alignment correction between the images.

### In-Vivo FF-OCT Axial Jitters Reduction and Compensation

Axial jitters (also referred to as axial measurement SOPs) are measured by using some of the SOPs transmitted during each measurement iteration.

During each measurement iteration SOPs are transmitted. One of the SOPs is a movement measurement SOP. Other SOPs are solely used for generating the depth information. It should be noted that during a single measurement iteration there may be two or more movement measurement SOPs.

For example:
(a) Assuming that is a need to illuminate a region of an in-vivo tissue with thirty seven different (narrowband) wavelength ranges.
(b) Assuming that adjacent measurement iteration SOPs should be spaced apart from each other by up to ten SOPs.

Table 1 illustrates an example of fulfilling these constraints. The SOP denoted 1 has a first wavelength range and is the movement measurement SOP. The second till 37^{th} SOPs are used for depth measurement and not for movement measurement.

TABLE 1

| | | | | | | | | | |
|---|---|---|---|---|---|---|---|---|---|
| 1 | 2 | 3 | 4 | 5 | 6 | 7 | 8 | 9 | 10 |
| 1 | 11 | 12 | 13 | 14 | 15 | 16 | 17 | 18 | 19 |
| 1 | 20 | 21 | 22 | 23 | 24 | 25 | 26 | 27 | 28 |
| 1 | 29 | 30 | 31 | 32 | 33 | 34 | 35 | 36 | 37 |

Yet for another example:
(a) Assuming that is a need to illuminate a region of an in-vivo tissue with thirty different (narrowband) wavelength ranges.
(b) Assuming that adjacent measurement iteration SOPs should be spaced apart from each other by up to five SOPs.

Table 2 illustrates two adjacent measurement iteration SOPs per measurement iteration—3 and 7.

TABLE 2

| | | | | | | | | |
|---|---|---|---|---|---|---|---|---|
| 1 | 2 | 3 | 4 | 5 | 6 | 7 | 8 | 9 |
| 10 | 11 | 3 | 12 | 13 | 14 | 7 | 15 | 16 |
| 17 | 18 | 3 | 19 | 20 | 21 | 7 | 22 | 23 |
| 24 | 25 | 3 | 26 | 27 | 38 | 7 | 29 | 30 |

It should be noted that the movement measurement SOP may be a SOP that is not the first SOP per transmission iteration and that there may be more than a single movement measurement SOP per measurement iteration.

If there are changes between the interference patterns detected during the different measurement iterations—the different may be processed to determine changes of axial distance between one measurement iteration to another.

According to an embodiment, the SOPs transmitted during different measurement iterations share an axial movement measurement SOP of a same wavelength range.

According to an embodiment, the SOPs transmitted during different measurement iterations also includes other SOPs that differ from each other by wavelength range.

According to an embodiment, the first SOPs transmitted during a first measurement iteration includes first other SOPs, wherein second SOPs transmitted during a second measurement iteration includes second other SOPs, wherein the first other SOPs differ by wavelength ranges from the second other SOPs.

According to an embodiment, the processing of the signals includes:
(a) Processing signals related to axial movement measurement SOPs of different measurement iterations to compensate for axial movements occurring between one measurement iteration to another
(b) Based on the compensation, processing signals related to other pulses of the different measurement iterations to provide the depth information regarding the region of the in-vivo tissue.

According to an embodiment, the method includes transmitting between two and twelve SOPs in each measurement iteration.

### Jitter Compensation Using OPL Compensation Component.

According to an embodiment, the measurement iterations are executed during a period of time; wherein the method further includes performing, during the period of time and by a optical distance measurement device, a plurality of distance change measurements to determine changes in the axial distance between the region and an axial optical distance measurement device. An example is illustrated in FIG. 25.

According to an embodiment, the method further includes compensating for the changes by introducing a delay at one of an arms of an interferometer.

According to an embodiment, the measurement iterations are executed during a period of time; wherein the method further includes performing, during the period of time and by an optical measurement device, a plurality of distance change measurements to determine changes in a distance between the region and an optical distance measurement device.

According to an embodiment, the method further includes compensating for the changes by introducing a delay at one of an arms of an interferometer.

According to an embodiment, the compensating includes mechanically setting a value of the delay.

According to an embodiment, the compensating includes mechanically moving two parts of a slider wedge in relation to each other to set a value of the delay

According to an embodiment, the compensating includes setting a value of the delay by controlling a refraction index of an adjustable refraction index delay unit.

According to an embodiment, the compensating includes setting a value of the delay by controlling a refraction index of a liquid crystal delay unit.

According to an embodiment, the compensating includes setting a value of the delay by controlling a refraction index of an electro-optical material.

According to an embodiment, the compensating includes setting a value of the delay by controlling a refraction index of a Pockels cell.

According to an embodiment, the distance change measurements includes transmitting radiation by the optical distance measurement device.

According to an embodiment, the method includes transmitting the radiation by the optical distance measurement device towards another in-vivo tissue that is in known spatial relationship with the region of the in-vivo tissue.

According to an embodiment, the other in-vivo tissue is located above or below or to side of the region of the in-vivo tissue. See, for example FIGS. 24A and 24B.

According to an embodiment, the method includes transmitting the radiation by the optical distance measurement device without utilizing any optical component of the interferometer.

According to an embodiment, the method includes transmitting the radiation by the optical distance measurement device while using at least one optical component that is shared with the interferometer.

According to an embodiment, the providing SOPs from the swept radiation source occurs at different times from an illuminating of the region by the optical distance measurement device.

According to an embodiment, the SOPs from the swept radiation source are of a different wavelength range than wavelength ranges of pulses transmitted from the optical distance measurement device.

According to an embodiment, the method includes performing a plurality of measurement sessions.

According to an embodiment, the different measurement sessions are associated with different combination of at least two parameters out of (a) a wavelength range of the swept illumination source; (b) the phase shifts introduced to pulses of a SOP in the selected arm of the interferometer, and (c) a focusing position of the sensing arm that corresponds to an optical path length of the reference arm.

### Slice Aggregation FF-SS-OCT

According to an embodiment, the measurement session is a first measurement session and the region of the in-vivo tissue is a first region of the in-vivo tissue, wherein the method includes preforming a second measurement sessions that includes performing measurement iterations of a second region of the in-vivo tissue, using the swept illumination source; wherein the second region and the first region partially overlap, wherein the second region is located either above the first region or below the first region.

According to an embodiment, the method includes generating depth information regarding a portion of the in-vivo tissue based on the information generated during the first measurement session and during the second measurement session.

According to an embodiment, the generating of the depth information regarding the portion uses overlap information regarding an overlap sub-region between the first region and the second region to align information obtained during the first measurement session with information obtained during the second measurement session.

According to an embodiment there are more than two slices (for example there are M slices, and there is an overlap region between some or all pairs of consecutive slices).

### OCT Angiography.

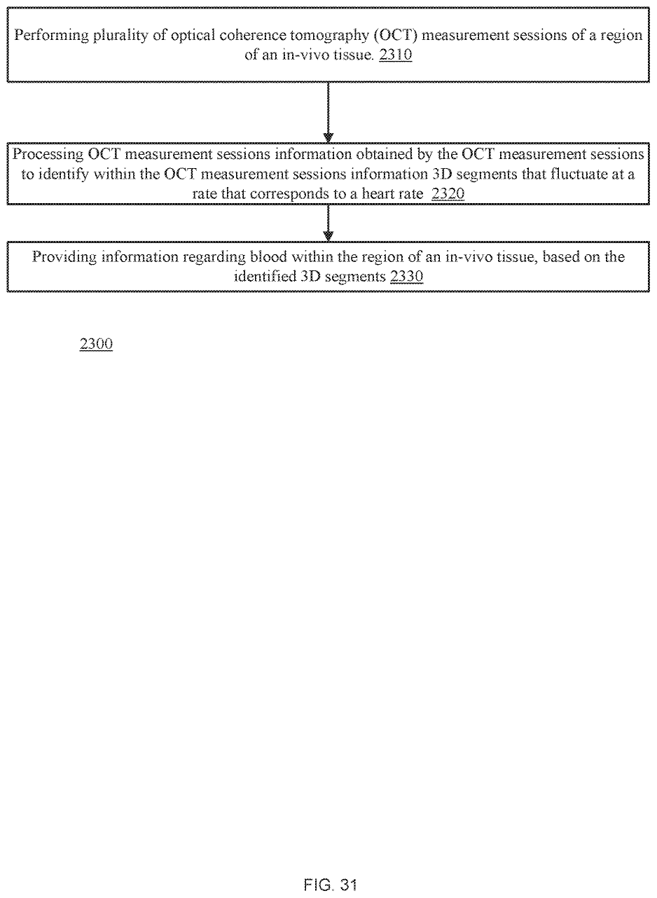

FIG. 31 illustrates an example of method 3200 for optical coherence tomography angiography (OCTA).

According to an embodiment, method 3200 includes step 3210 of performing plurality of optical coherence tomography (OCT) measurement sessions of a region of an in-vivo tissue; wherein the performing of the OCT measurements sessions includes illuminating, by aera illumination, the region and collecting interference patterns resulting from the illumination by a two dimensional (D) array of radiation sensors.

According to an embodiment, step 3210 is followed by step 3220 of processing OCT measurement sessions information obtained by the OCT measurement sessions to identify within the OCT measurement sessions information 3D segments that fluctuate at a rate that corresponds to a heart rate. The heart rate may range between 40-180 heat beats per minute, and the like.

According to an embodiment, step 3220 is followed by step 3230 of providing information regarding blood within the region of an in-vivo tissue, based on the identified 3D segments.

According to an embodiment, the OCT measurement sessions information includes three dimensional (3D) reflectively maps based on outcomes of the OCT measurement sessions.

According to an embodiment, the processing includes aligning the OCT measurement sessions information.

According to an embodiment, the processing includes enhancing voxels with high temporal fluctuations

According to an embodiment, the processing includes enhancing temporal harmonic variation that correlates to the heart rate or harmonics of the heart rate.

According to an embodiment, the processing includes enhancing neighboring voxels that fluctuate a same rate and phase as voxels that represent blood vessels.

According to an embodiment, the processing includes suppressing noise data that fluctuates randomly in time or space.

According to an embodiment, the method includes determining blood flow.

According to an embodiment, the information about blood includes a location of blood vessels.

According to an embodiment, the information about blood is information about blood flow.

According to an embodiment, the information about blood flow includes information related to a direction of the blood flow.

According to an embodiment, the information about blood flow includes information related to a speed of the blood flow.

According to an embodiment, the information about the blood flow is deducted based on phase differences between detected signal fluctuation of adjacent 3D segments.

According to an embodiment, the method includes generating a 3D map of blood vessels.

According to an embodiment, the method includes generating a 3D map that illustrates positioned of blood vessels and blood flow propagation information. See, for example FIG. 29.

Depth information regarding a region is indicative of the content of a partially transparent region of a sample or top surface 3d mapping of non transparent region of a sample. The depth information may be represented as a 3D map but other representations of the depth information may be provided—for example a collection of 2D maps, edge information, material information, and the like.

### Abbreviations

OCT—Optical coherence tomography.
OCTA—OCT angiography.
TD-OCT—Time Domain OCT.
SD-OCT—Spectral Domain OCT.
FF-OCT—Full Field OCT.
SS-OCT—Swept radiation source OCT.
FF-SS-OCT—Full Field SS-OCT
FF-TD-OCT—Full Field TD-OC
TOF—Time Of Flight.
SLD—Super-Luminescent Diode
AO—Adaptive Optics.
AD—Alzheimer Disease.
PD—Photodiode.
FFT—Fast Fourier Transform.
CNS—Central Nervous System.
CSF—Cerebrospinal fluid.
AMD—Age-related Macular Degeneration.
NA—Numerical Aperture.
DOF—Depth of Focus.
SL—Slit Lamp.
FLIO—Fluorescence Lifetime Imaging Ophthalmoscopy.
FLIM—Fluorescence Lifetime Imaging Microscopy.
CFA—Color Filter Array.
SL—Slit-Lamp.
EEG—Electroencephalogram.
FNIRS—Functional Near-Infrared Spectroscopy.
Aβ—Amyloid Beta.
ML—Machine Learning
IRF—Instrument Response Functions
TCSPC—Time-Correlated Single Photon Counting

The terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the disclosure described herein are, for example, capable of operation in other orientations than those illustrated or otherwise described herein. The connections as discussed herein may be any type of connection suitable to transfer signals from or to the respective nodes, units, or devices, for example via intermediate devices. Accordingly, unless implied or stated otherwise, the connections may for example be direct connections or indirect connections. The connections may be illustrated or described in reference to be a single connection, a plurality of connections, unidirectional connections, or bidirectional connections. However, different embodiments may vary the implementation of the connections. For example, separate unidirectional connections may be used rather than bidirectional connections and vice versa. Also, plurality of connections may be replaced with a single connection that transfers multiple signals serially or in a time multiplexed manner. Likewise, single connections carrying multiple signals may be separated out into various different connections carrying subsets of these signals. Therefore, many options exist for transferring signals.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above-described operations are merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also, for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

However, other modifications, variations and alternatives are also possible. The specifications and drawings are, accordingly, to be regarded in an illustrative rather than in a restrictive sense. In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to embodiments of the disclosure s containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the embodiments of the disclosure have been illustrated and described herein, many modifications, substitutions, changes, and equivalents will now occur to those of ordinary skill in the art. It is, therefore, to be understood that the appended claims are intended to cover all such modifications and changes as fall within the true spirit of the embodiments of the disclosure.

Any reference to “comprising” should be applied, mutatis mutandis, to “consisting essentially of”.

Any reference to “comprising” should be applied, mutatis mutandis, to “consisting”.

We claim:

1. A method for optical coherence tomography (OCT), the method comprising:
performing a measurement session that comprises performing measurement iterations of a region of an in-vivo tissue, using a swept illumination source;
wherein each measurement iteration comprises:
providing sets of pulses (SOPs) of radiation to an interferometer; wherein pluses of each SOP have a same wavelength range, and at least some of the SOPs differ from each other by their wavelength range;
splitting, by the interferometer, the SOPs to a first path SOPs and a second path SOPs;
generating by the interferometer, interference patterns that represents an interference between (i) the first path SOPs, following a propagation of the first path SOPs along a first path and an interaction of the first path SOPs with the region of the in-vivo-tissue, and (ii) the second path SOPs, following a propagation of the second path SOPs though a second path and an interaction of the second path SOPs with a reference arm of the interferometer;
wherein a selected path out of the first path and the second path introduces phase changes between pulses of a same set; and
generating signals indicative of the interference pattern by two dimensional (2D) array of sensing related elements of a sensing unit; and
processing the signals;
wherein the processing of the signals comprises determining depth information regarding the region of an in-vivo tissue.

2. The method according to claim 1, wherein at least one set of pulses comprises three pulses.

3. The method according to claim 1, wherein the interferometer belongs to a hand held OCT device.

4. The method according to claim 1, comprising sensing different pulses of a set of pulses by different accumulation elements of a 2D array of the sensing unit.

5. The method according to claim 1, comprising introduces a lateral alignment correction between the images.

6. The method according to claim 5, wherein SOPs transmitted during different measurement iterations share an axial movement measurement SOP of a same wavelength range.

7. The method according to claim 6, wherein the SOPs transmitted during different measurement iterations also comprises other SOPs that differ from each other by wavelength range.

8. The method according to claim 7, wherein first SOPs transmitted during a first measurement iteration comprise first other SOPs, wherein second SOPs transmitted during a second measurement iteration comprise second other SOPs, wherein the first other SOPs differ by wavelength ranges from the second other SOPs.

9. The method according to claim 7, wherein the processing of the signals comprises:
processing signals related to axial movement measurement SOPs of different measurement iterations to compensate for axial movements occurring between one measurement iteration to another; and
based on the compensation, processing signals related to other pulses of the different measurement iterations to provide the depth information regarding the region of the in-vivo tissue.

10. The method according to claim 6, comprising transmitting between two and twelve SOPs in each measurement iteration.

11. The method according to claim 5, wherein the measurement iterations are executed during a period of time; wherein the method further comprises performing, during the period of time and by an optical distance measurement device, a plurality of distance change measurements to determine changes in the axial distance between the region and an axial optical distance measurement device.

12. The method according to claim 11, wherein the method further comprises compensating for the changes by introducing a delay at one of an arms of an interferometer.

13. The method according to claim 1, wherein the measurement iterations are executed during a period of time; wherein the method further comprises performing, during the period of time and by an optical measurement device, a plurality of distance change measurements to determine changes in a distance between the region and an optical distance measurement device.

14. The method according to claim 13, wherein the method further comprises compensating for the changes by introducing a delay at one of an arms of an interferometer.

15. The method according to claim 13, wherein the compensating comprises mechanically setting a value of the delay.

16. The method according to claim 13, wherein the compensating comprises mechanically moving two parts of a slider wedge in relation to each other to set a value of the delay

17. The method according to claim 13, wherein the compensating comprises setting a value of the delay by controlling a refraction index of an adjustable refraction index delay unit.

18. The method according to claim 13, wherein the compensating comprises setting a value of the delay by controlling a refraction index of a liquid crystal delay unit.

19. The method according to claim 13, wherein the compensating comprises setting a value of the delay by controlling a refraction index of an electro-optical material.

20. The method according to claim 13, wherein the compensating comprises setting a value of the delay by controlling a refraction index of a Pockels cell.

21. The method according to claim 13, wherein the distance change measurements comprise transmitting radiation by the optical distance measurement device.

22. The method according to claim 21, wherein the method comprises transmitting the radiation by the optical distance measurement device towards another in-vivo tissue that is in known spatial relationship with the region of the in-vivo tissue.

23. The method according to claim 21, wherein the other in-vivo tissue is located above or below or to side of the region of the in-vivo tissue.

24. The method according to claim 13, wherein the method comprises transmitting the radiation by the optical distance measurement device without utilizing any optical component of the interferometer.

25. The method according to claim 13, wherein the method comprises transmitting the radiation by the optical distance measurement device while using at least one optical component that is shared with the interferometer.

26. The method according to claim 13, wherein the providing SOPs from the swept radiation source occurs at different times from an illuminating of the region by the optical distance measurement device.

27. The method according to claim 13, wherein the SOPs from the swept radiation source are of a different wavelength range than wavelength ranges of pulses transmitted from the optical distance measurement device.

28. The method according to claim 1, comprising performing a plurality of measurement sessions.

29. The method according to claim 28, wherein different measurement sessions are associated with different combination of at least two parameters out of (a) a wavelength range of the swept illumination source; (b) the phase shifts introduced to pulses of a SOP in the selected arm of the interferometer, and (c) a focusing position of the sensing arm that corresponds to an optical path length of the reference arm.

30. The method according to claim 1, wherein the measurement session is a first measurement session and the region of the in-vivo tissue is a first region of the in-vivo tissue, wherein the method comprises preforming a second measurement sessions that comprises performing measurement iterations of a second region of the in-vivo tissue, using the swept illumination source; wherein the second region and the first region partially overlap, wherein the second region is located either above the first region or below the first region.

31. The method according to claim 30, comprising generating depth information regarding a portion of the in-vivo tissue based on the information generated during the first measurement session and during the second measurement session.

32. The method according to claim 31, wherein the generating of the depth information regarding the portion uses overlap information regarding an overlap sub-region between the first region and the second region to align information obtained during the first measurement session with information obtained during the second measurement session.

33. A method for optical coherence tomography angiography (OCTA), the method comprises:
performing optical coherence tomography (OCT) measurement sessions of a region of an in-vivo tissue; wherein the performing of the OCT measurements sessions comprises illuminating, by aera illumination, the region and collecting interference patterns resulting from the illumination by a two dimensional (2D) array of radiation sensors;
processing OCT measurement sessions information obtained by the OCT measurement sessions to identify within the OCT measurement sessions information 3D segments that fluctuate at a rate that corresponds to a heart rate; and
providing information regarding blood within the region of an in-vivo tissue, based on the identified 3D segments.

34. The method according to claim 33 wherein the OCT measurement sessions information comprise three dimensional (3D) reflectively maps based on outcomes of the OCT measurement sessions.

35. The method according to claim 34, wherein the processing comprises aligning the OCT measurement sessions information.

36. The method according to claim 33, wherein the processing comprises enhancing 3D segments with high temporal fluctuations

37. The method according to claim 33, wherein the processing comprises enhancing temporal harmonic variation that correlates to the heart rate or harmonics of the heart rate.

38. The method according to claim 33, wherein the processing comprises enhancing neighboring voxels that fluctuate a same rate and phase as voxels that represent blood vessels.

39. The method according to claim 33, wherein the processing comprises suppressing noise data that fluctuates randomly in time or space.

40. The method according to claim 33, comprising determining blood flow.

41. The method according to claim 33, wherein the information about blood comprises a location of blood vessels.

42. The method according to claim 33, wherein the information about blood is information about blood flow.

43. The method according to claim 42, wherein the information about blood flow comprises information related to a direction of the blood flow.

44. The method according to claim 42, wherein the information about blood flow comprises information related to a speed of the blood flow.

45. The method according to claim 42, wherein the information about the blood flow is deducted based on phase differences between detected signal fluctuation of adjacent 3D segments.

46. The method according to claim 42, comprising generating a 3D map of blood vessels.

47. The method according to claim 42, comprising generating a 3D map that illustrates positioned of blood vessels and blood flow propagation information.

48. A non-transitory computer readable medium for optical coherence tomography (OCT), the non-transitory computer readable medium that stores instructions for:
performing a measurement session that comprises performing measurement iterations of a region of an in-vivo tissue, using a swept illumination source;
wherein each measurement iteration comprises:
providing sets of pulses (SOPs) of radiation to an interferometer; wherein pluses of each SOP have a same wavelength range, and at least some of the SOPs differ from each other by their wavelength range;
splitting, by the interferometer, the SOPs to a first path SOPs and a second path SOPs;
generating by the interferometer, interference patterns that represents an interference between (i) the first path SOPs, following a propagation of the first path SOPs along a first path and an interaction of the first path SOPs with the region of the in-vivo-tissue, and (ii) the second path SOPs, following a propagation of the second path SOPs though a second path and an interaction of the second path SOPs with a reference arm of the interferometer;
wherein a selected path out of the first path and the second path introduces phase changes between pulses of a same set; and
generating signals indicative of the interference pattern by two dimensional (2D) array of sensing related elements of a sensing unit; and
processing the signals;
wherein the processing of the signals comprises determining depth information regarding the region of an in-vivo tissue.

49. A non-transitory computer readable medium for optical coherence tomography angiography (OCTA), the non-transitory computer readable medium that stores instructions for:
performing optical coherence tomography (OCT) measurement sessions of a region of an in-vivo tissue; wherein the performing of the OCT measurements sessions comprises illuminating, by aera illumination, the region and collecting interference patterns resulting from the illumination by a two dimensional (2D) array of radiation sensors;
processing OCT measurement sessions information obtained by the OCT measurement sessions to identify within the OCT measurement sessions information 3D segments that fluctuate at a rate that corresponds to a heart rate; and
providing information regarding blood within the region of an in-vivo tissue, based on the identified 3D segments.

50. The non-transitory computer readable medium according to claim 49, wherein the information regarding blood comprises information about a blood flow.

51. A device for optical coherence tomography (OCT), the device comprising:
an interferometer;
a swept radiation source;
a sensing unit that comprises a two dimensional (2D) array of sensing related elements;
a processor; and
a controller that is configured to control a performing of a measurement session that comprises performing measurement iterations of a region of an in-vivo tissue, using a swept illumination source;
wherein during each measurement iteration:
the swept radiation source is configured to provide sets of pulses (SOPs) of radiation to the interferometer; wherein pluses of each SOP have a same wavelength range, and at least some of the SOPs differ from each other by their wavelength range;
the interferometer is configured to:
split the SOPs to a first path SOPs and a second path SOPs;
generate interference patterns that represents an interference between (i) the first path SOPs, following a propagation of the first path SOPs along a first path and an interaction of the first path SOPs with the region of the in-vivo-tissue, and (ii) the second path SOPs, following a propagation of the second path SOPs though a second path and an interaction of the second path SOPs with a reference arm of the interferometer; wherein a selected path out of the first path and the second path introduces phase changes between pulses of a same set; and
the 2D array of sensing related elements is configured to generate signals indicative of the interference pattern;
wherein the processor is configured to process the signals; and to determine depth information regarding the region of an in-vivo tissue.

52. The device according to claim 51, wherein at least one set of pulses comprises three pulses.

53. The device according to claim 51, wherein the interferometer belongs to a hand held OCT device.

54. The device according to claim 51, wherein the 7D array of sensing related elements comprises different accumulation elements configured to sense different pulses of a set of pulses.

55. The device according to claim 51, wherein the processor is configured to introduce a lateral alignment correction between the images.

56. The device according to claim 51, wherein SOPs transmitted during different measurement iterations share an axial movement measurement SOP of a same wavelength range.

57. The device according to claim 56, wherein the SOPs transmitted during different measurement iterations also comprise other SOPs that differ from each other by wavelength range.

58. The device according to claim 57, wherein first SOPs transmitted during a first measurement iteration comprise first other SOPs, wherein second SOPs transmitted during a second measurement iteration comprise second other SOPs, wherein the first other SOPs differ by wavelength ranges from the second other SOPs.

59. The device according to claim 57, wherein the processor is further configured to:
process signals related to axial movement measurement SOPs of different measurement iterations to compensate for axial movements occurring between one measurement iteration to another; and
based on the compensation, process signals related to other pulses of the different measurement iterations to provide the depth information regarding the region of the in-vivo tissue.

60. The device according to claim 56, wherein the steps illumination source is configured to transmit between two and twelve SOPs in each measurement iteration.

61. The device according to claim 56, wherein the measurement iterations are executed during a period of time; wherein the device further comprises a optical distance measurement device that is configured to perform, during the period of time, a plurality of distance change measurements to determine changes in the axial distance between the region and an axial optical distance measurement device.

62. The device according to claim 61, wherein the device further comprises a distance compensating unit configured to compensate for the changes by introducing a delay at one of an arms of an interferometer.

63. The device according to claim 61, wherein the measurement iterations are executed during a period of time; wherein the processor is further configured to perform, during the period of time, a plurality of distance change measurements to determine changes in a distance between the region and an optical distance measurement device.

64. The device according to claim 63, wherein the device further comprises a distance compensating unit that is configured to compensate for the changes by introducing a delay at one of an arms of an interferometer.

65. The device according to claim 63, wherein the distance compensating unit is configured to compensate by mechanically setting a value of the delay.

66. The device according to claim 63, wherein the distance compensating unit is configured to compensate by mechanically moving two parts of a slider wedge in relation to each other to set a value of the delay

67. The device according to claim 63, wherein the distance compensating unit is configured to compensate by controlling a refraction index of an adjustable refraction index delay unit.

68. The device according to claim 63, wherein the distance compensating unit is configured to compensate by controlling a refraction index of a liquid crystal delay unit.

69. The device according to claim 63, wherein the wherein the distance compensating unit is configured to compensate by controlling a refraction index of an electro-optical material.

70. The device according to claim 63, wherein the distance compensating unit is configured to compensate by controlling a refraction index of a Pockels cell.

71. The device according to claim 63, wherein the optical distance measurement device is configured to transmit radiation.

72. The device according to claim 71, wherein the optical distance measurement device is configured to transmit the radiation towards another in-vivo tissue that is in known spatial relationship with the region of the in-vivo tissue.

73. The device according to claim 71, wherein the other in-vivo tissue is located above or below or to side of the region of the in-vivo tissue.

74. The device according to claim 13, wherein the optical distance measurement device is configured to transmit the radiation without utilizing any optical component of the interferometer.

75. The device according to claim 13, wherein the optical distance measurement device is configured to transmit the radiation while using at least one optical component that is shared with the interferometer.

76. The device according to claim 13, wherein the wherein the optical distance measurement device is configured to transmit the radiation at time windows that do not overlap a timing of transmission of the swept radiation source.

77. The device according to claim 13, wherein the SOPs from the swept radiation source are of a different wavelength range than wavelength ranges of pulses transmitted from the optical distance measurement device.

78. The device according to claim 1, configured to perform a plurality of measurement sessions.

79. The device according to claim 78, wherein different measurement sessions are associated with different combination of at least two parameters out of (a) a wavelength range of the swept illumination source; (b) the phase shifts introduced to pulses of a SOP in the selected arm of the interferometer, and (c) a focusing position of the sensing arm that corresponds to an optical path length of the reference arm.

80. The device according to claim 1, wherein the measurement session is a first measurement session and the region of the in-vivo tissue is a first region of the in-vivo tissue, wherein the device is configured to preform a second measurement sessions that comprises performing measurement iterations of a second region of the in-vivo tissue, using the swept illumination source; wherein the second region and the first region partially overlap, wherein the second region is located either above the first region or below the first region.

81. The device according to claim 80, wherein the processor is configured to generate depth information regarding a portion of the in-vivo tissue based on the information generated during the first measurement session and during the second measurement session.

82. The device according to claim 81, wherein the processor is configured to generate of the depth information regarding the portion uses overlap information regarding an overlap sub-region between the first region and the second region to align information obtained during the first measurement session with information obtained during the second measurement session.

83. A device for optical coherence tomography angiography (OCTA), the device comprises:
an interferometer;
a radiation source;
a sensing unit that comprises a two dimensional (2D) array of sensing related elements;
a processor; and
a controller;
wherein the device is configured to perform optical coherence tomography (OCT) measurement sessions of a region of an in-vivo tissue; wherein the performing of the OCT measurements sessions comprises illuminating the region, by aera illumination, under a control of the controller, and using the radiation source; feeding radiation from the region to the interferometer, generating interference patterns by the interferometer, and collecting interference patterns resulting from the illumination by the 7D array of radiation sensors;
wherein the processor is configured to
process OCT measurement sessions information obtained by the OCT measurement sessions to identify within the OCT measurement sessions information 8D segments that fluctuate at a rate that corresponds to a heart rate; and
provide information regarding blood within the region of an in-vivo tissue, based on the identified 8D segments.

84. The device according to claim 83 wherein the OCT measurement sessions information comprise three dimensional (3D) reflectively maps based on outcomes of the OCT measurement sessions.

85. The device according to claim 84, wherein the processor is configured to align the OCT measurement sessions information.

86. The device according to claim 83, wherein the processor is configured to enhance voxels with high temporal fluctuations

87. The device according to claim 83, wherein the processor is configured to enhance temporal harmonic variation that correlates to the heart rate or harmonics of the heart rate.

88. The device according to claim 83, wherein the processor is configured to enhance neighboring voxels that fluctuate a same rate and phase as voxels that represent blood vessels.

89. The device according to claim 83, wherein the processor is configured to suppress noise data that fluctuates randomly in time or space.

90. The device according to claim 83, wherein the processor is configured to determine blood flow.

91. The device according to claim 83, wherein the information about blood comprises a location of blood vessels.

92. The device according to claim 83, wherein the information about blood is information about blood flow.

93. The device according to claim 92, wherein the information about blood flow comprises information related to a direction of the blood flow.

94. The device according to claim 92, wherein the information about blood flow comprises information related to a speed of the blood flow.

95. The device according to claim 92, wherein the processor is configured to deduct the blood flow based on phase differences between detected signal fluctuation of adjacent 8D segments.

96. The device according to claim 92, wherein the processor is configured to generate a 8D map of blood vessels.

97. The device according to claim 92, wherein the processor is configured to generate a 8D map that illustrates positioned of blood vessels and blood flow propagation information.

98. The device according to claim 51, wherein the radiation source is a swept radiation source and during each measurement iteration: (a) the swept radiation source is configured to provide sets of pulses (SOPs) of radiation to the interferometer; wherein pluses of each SOP have a same wavelength range, and at least some of the SOPs differ from each other by their wavelength range; (b) the interferometer is configured to: split the SOPs to a first path SOPs and a second path SOPs; generate interference patterns that represents an interference between (i) the first path SOPs, following a propagation of the first path SOPs along a first path and an interaction of the first path SOPs with the region of the in-vivo-tissue, and (ii) the second path SOPs, following a propagation of the second path SOPs though a second path and an interaction of the second path SOPs with a reference arm of the interferometer; wherein a selected path out of the first path and the second path introduces phase changes between pulses of a same set; and the 2D array of sensing related elements is configured to generate signals indicative of the interference pattern.
